SEQUENCE LISTING (69 sequences)

SEQ ID NO: 1 - DNA, 20 bp, Oryza sativa
gcggtaaccaacttcgacat

SEQ ID NO: 2 - DNA, 20 bp, Oryza sativa
ctgcattctcacttcggtca

SEQ ID NO: 3 - DNA, 19 bp, Oryza sativa
ccgcgctctccgtgttctc

SEQ ID NO: 4 - DNA, 18 bp, Oryza sativa
gtgctgcgccgcctcctt

SEQ ID NO: 5 - DNA, 20 bp, Oryza sativa
tcggatggttcgcggcgaag

SEQ ID NO: 6 - DNA, 20 bp, Oryza sativa
gtcgtacatgcgccggatgg

SEQ ID NO: 7 - DNA, 21 bp, Oryza sativa
ttctgccaaagccaccgattc

SEQ ID NO: 8 - DNA, 20 bp, Oryza sativa
acggatgcgacgccaatacg

SEQ ID NO: 9 - DNA, 21 bp, Oryza sativa
cctccatccagtgtgaccatc

SEQ ID NO: 10 - DNA, 20 bp, Oryza sativa
agcccatgttcccctcgccc

SEQ ID NO: 11 - DNA, 20 bp, Oryza sativa
cggtgcctcacagtgctcca

SEQ ID NO: 12 - DNA, 21 bp, Oryza sativa
aacaccataccgtcctggatg

SEQ ID NO: 13 - DNA, 20 bp, Oryza sativa
caccagcattggcgccgaca

SEQ ID NO: 14 - DNA, 20 bp, Oryza sativa
gcgttctcagctgctgcgtg

SEQ ID NO: 15 - DNA, 20 bp, Oryza eichingeri
gagctgaaataggcccgtac

SEQ ID NO: 16 - DNA, 21 bp, Oryza eichingeri
gaggattctttgttgccatcg

SEQ ID NO: 17 - DNA, 22 bp, Oryza eichingeri
ataggcccgtacttcaatggtt

SEQ ID NO: 18 - DNA, 20 bp, Oryza eichingeri
gagaaccagccatccatccg

SEQ ID NO: 19 - DNA, 22 bp, Oryza eichingeri
gctgctaccaaacaattcacaa

SEQ ID NO: 20 - DNA, 22 bp, Oryza eichingeri
actccagctgaagcagatggtt

SEQ ID NO: 21 - DNA, 29 bp, Oryza sativa
gctctagaatggcagggtctcttgccgcc

SEQ ID NO: 22 - DNA, 29 bp, Oryza sativa
cggaattcctaggctaacttttcactctg

SEQ ID NO: 23 - DNA, 28 bp, Oryza sativa
gctctagaatggctggttctcttgcggc

SEQ ID NO: 24 - DNA, 28 bp, Oryza sativa
cggaattctcatgcactctcagctggga

SEQ ID NO: 25 - DNA, 29 bp, Oryza sativa
gctctagaatggcagggtctcttgccgcc

SEQ ID NO: 26 - DNA, 30 bp, Oryza sativa
cggaattcttacgcgttctcagctgctgcg

SEQ ID NO: 27 - DNA, 28 bp, Hordeum vulgare
gctctagactgcaggtcaacggatcctt

SEQ ID NO: 28 - DNA, 28 bp, Hordeum vulgare
gctctagaagttctatttcactcagggt

SEQ ID NO: 29 - DNA, 28 bp, Oryza eichingeri
tttctagaatggctggttctcttgcggc

SEQ ID NO: 30 - DNA, 28 bp, Oryza eichingeri
atgaattcttgccggataaactacagaa

SEQ ID NO: 31 - DNA, 20 bp, Oryza sativa
gtacatgtaggtcttgttta

SEQ ID NO: 32 - DNA, 20 bp, Oryza sativa
cttctagctgatgctgcagg

SEQ ID NO: 33 - DNA, 20 bp, Oryza sativa
acagaaatttcgctggccat

SEQ ID NO: 34 - DNA, 20 bp, Oryza sativa
ctggcaattcaccggttgtg

SEQ ID NO: 35 - DNA, 20 bp, Oryza sativa
ttctcgtatcctagcccata

SEQ ID NO: 36 - DNA, 20 bp, Oryza sativa
cttctagctgatgctgcagg

SEQ ID NO: 37 - DNA, 21 bp, Oryza eichingeri
acagaaatttcgctggccatg

SEQ ID NO: 38 - DNA, 20 bp, Oryza eichingeri
ccacagacactcaaattctc

SEQ ID NO: 39 - DNA, 21 bp, Oryza eichingeri
acagaaatttcgctggccatg

SEQ ID NO: 40 - DNA, 20 bp, Oryza eichingeri
ccacagacactcaaattctc

SEQ ID NO: 41 - DNA, 897 bp, Oryza sativa
atggcagggtctcttgccgcctcagcattcttcccaggtccaggctcatctcctgcagcatcagctagaagctccaagaatgctgctgttaccggcgaattgccggagaatttgagtgtctgtggcattgtcgcaaagcctaacccacctcctgcagccatgcaagtaaaggcacaggctcaaacccttcccaaggttaatggtacgaaggttaacctcaagacggtgaagcctgacatggaggaaacggtgcctcacagtgctccaaagacgttctataaccaactgccggattggagcatgcttcttgcggctattacaaccatcttcctcgccgcagagaagcagtggacactgcttgattggaagccgaagaaacctgacatgcttgttgacacatttggctttggtaggatcatccaggacggtatggtgtttaggcagaacttcatgattcggtcctacgagattggcgctgatcgtacagcttctatagagacattgatgaatcatttacaggaaacggctcttaaccatgtaaggactgctggtcttcttggagatggttttggggctacaccggagatgagcaaacggaacttgatatgggttgtcagcaaaatccagcttcttgttgagcaataccccgcatggggagatatggttcaagttgacacatgggtcgctgctgctggcaaaaatggcatgcgtcgagactggcatgttcgtgactacaactctggccgaacaatcttgagagctacaagtgtttgggtgatgatgcacaagaaaactagaagactttcaaaaatgccagatgaagttagagctgaaataggcccatatttcaatgaccgttcagctataacagaggagcagagtgaaaagttagcctag

SEQ ID NO: 42 - protein, 298 aa, Oryza sativa
MAGSLAASAFFPGPGSSPAASARSSKNAAVTGELPENLSVCGIVAKPNPPPAAMQVKAQAQTLPKVNGTKVNLKTVKPDMEETVPHSAPKTFYNQLPDWSMLLAAITTIFLAAEKQWTLLDWKPKKPDMLVDTFGFGRIIQDGMVFRQNFMIRSYEIGADRTASIETLMNHLQETALNHVRTAGLLGDGFGATPEMSKRNLIWVVSKIQLLVEQYPAWGDMVQVDTWVAAAGKNGMRRDWHVRDYNSGRTILRATSVWVMMHKKTRRLSKMPDEVRAEIGPYFNDRSAITEEQSEKLA

SEQ ID NO: 43 - DNA, 1284 bp, Oryza sativa
atggctggttctcttgcggcgtctgcattcttccctgtcccagggtcttcccctgcagcttcggctagaagctctaagaacacaaccggtgaattgccagagaatttgagtgtccgcggaatcgtcgcgaagcctaatccgtctccaggggccatgcaagtcaaggcgcaggcgcaagcccttcctaaggttaatggaaccaaggttaacctgaagactacaagcccagacaaggaggatataataccgtacactgctccgaagacattctataaccaattgccagactggagcatgcttcttgcagctgtcacgaccattttcctggcagctgagaagcagtggactctgcttgactggaagccgaagaagcctgacatgctggctgacacattcggctttggtaggatcatccaagacgggctggtgtttaggcaaaacttcttgattcggtcctacgagattggtgctgatcgtacagcttctattgagacattaatgaatcatttacaggaaacagctctgaaccatgtgaaaactgctggtctcttaggtgatggttttggtgctacgccggagatgagcaaacggaacttaatatgggttgtcagcaaaattcagcttcttgttgagcgatacccatcatggggagatatggtccaagttgacacatgggtagctgctgctggcaaaaatggcatgcgtcgagattggcatgttcggaactacaactctggtcaaacaatcttgagggctacaagtgtttgggtgatgatgaataagaacactagaagactttcaaaaatgccagatgaagttagagctgaaataggcccgtatttcaatggccgttctgctatatcagaggagcagggtgaaaagttgcctaagccagggaccacatttgatggcgctgctaccaaacaattcacaagaaaagggcttactccgaagtggagtgaccttgatgtcaaccagcatgtgaacaatgtgaagtatattggttggatacttgagagtgctccaatttcgatactggagaagcacgagcttgcaagcatgaccttggattacaggaaggagtgtggccgtgacagtgtgcttcagtcgcttaccgctgtttcaggtgaatgcgatgatggcaacacagaatcctccatccagtgtgaccatctgcttcagctggagtccggagcagacattgtgaaggctcacacagagtggcgaccgaagcgagctcagggcgaggggaacatgggctttttcccagctgagagtgcatga

SEQ ID NO: 44 - protein, 427 aa, Oryza sativa
MAGSLAASAFFPVPGSSPAASARSSKNTTGELPENLSVRGIVAKPNPSPGAMQVKAQAQALPKVNGTKVNLKTTSPDKEDIIPYTAPKTFYNQLPDWSMLLAAVTTIFLAAEKQWTLLDWKPKKPDMLADTFGFGRIIQDGLVFRQNFLIRSYEIGADRTASIETLMNHLQETALNHVKTAGLLGDGFGATPEMSKRNLIWVVSKIQLLVERYPSWGDMVQVDTWVAAAGKNGMRRDWHVRNYNSGQTILRATSVWVMMNKNTRRLSKMPDEVRAEIGPYFNGRSAISEEQGEKLPKPGTTFDGAATKQFTRKGLTPKWSDLDVNQHVNNVKYIGWILESAPISILEKHELASMTLDYRKECGRDSVLQSLTAVSGECDDGNTESSIQCDHLLQLESGADIVKAHTEWRPKRAQGEGNMGFFPAESA

SEQ ID NO: 45 - DNA, 1278 bp, Oryza sativa
atggcagggtctcttgccgcctcagcattcttcccaggtccaggctcatctcctgcagcatcagctagaagctccaagaatgctgctgttaccggcgaattgccggagaatttgagtgtccgtggcattgtcgcaaagcctaacccacctcctgcagccatgcaagtaaaggcacaggctcaaacccttcccaaggttaatggtacgaaggttaacctcaagacggtgaagcctgacatggaggaaacggtgccttacagtgctccaaagacgttctataaccaactgccggattggagcatgcttcttgcggctattacaaccatcttccttgccgcagagaagcagtggacactgcttgattggaagccaaagaaacctgacatgcttgttgacacatttggctttggtaggattatccaggacggtatggtgtttaggcagaacttcatgattcggtcctacgagattggtgctgatcgtacagcttctatagagacattgatgaatcatttacaggaaacagctcttaaccatgtgaggactgctggtcttcttggagatggttttggggctacaccggagatgagcaaacggaacttgatatgggttgtcagcaaaatccagcttcttgttgagcaataccccgcatggggagatacggttcaagttgacacatgggttgctgctgctggcaaaaatggcatgcgtcgagactggcatgttcgtgactacaactctggccgaacaatcttgagagctacaagtgtttgggtgatgatgcacaagaaaactagaagactttcaaaaatgccagatgaagttagagctgaaataggcccatatttcaatgaccgttcagctataacagaggagcagagtgaaaagttagccaagacaggaaataaagttggtgatgatgctacagagcaattcataagaaaggggctcactcctagatggggtgacctcgatgtcaatcagcatgtgaacaatgttaaatatattgggtggatccttgagagtgctccaatttcagtactggagaagcatgagcttgcaagcatgaccctggattacaggaaggagtgtggtcgagacagcgtgctgcaatcacttaccaccgtgtcaggggaatgcaccagcattggcgccgacaagcaggcttctgccatccagtgcgaccatcttcttcagcttgagtcaggagctgatattgtgaaggcacacacagagtggcgaccaaagcgatcgcacgcagcagctgagaacgcgtaa

SEQ ID NO: 46 - protein, 425 aa, Oryza sativa
MAGSLAASAFFPGPGSSPAASARSSKNAAVTGELPENLSVRGIVAKPNPPPAAMQVKAQAQTLPKVNGTKVNLKTVKPDMEETVPYSAPKTFYNQLPDWSMLLAAITTIFLAAEKQWTLLDWKPKKPDMLVDTFGFGRIIQDGMVFRQNFMIRSYEIGADRTASIETLMNHLQETALNHVRTAGLLGDGFGATPEMSKRNLIWVVSKIQLLVEQYPAWGDTVQVDTWVAAAGKNGMRRDWHVRDYNSGRTILRATSVWVMMHKKTRRLSKMPDEVRAEIGPYFNDRSAITEEQSEKLAKTGNKVGDDATEQFIRKGLTPRWGDLDVNQHVNNVKYIGWILESAPISVLEKHELASMTLDYRKECGRDSVLQSLTTVSGECTSIGADKQASAIQCDHLLQLESGADIVKAHTEWRPKRSHAAAENA

SEQ ID NO: 47 - DNA, 1083 bp, Oryza eichingeri
atggctggttctcttgcggcgtctgcattcttccctagcccagggtcttcccctgcagcatcgactagaagttctaagaatacaaccagtgaattgccagagaatttgagtgtccgtggaatcgtcgcgaagcctaacccgcctccgggggccatgcaagtcaaggcgcaagcgcaagcccttcccaaggttaatggaaccaaggttaacctgaagactacaagcccagagaaggaggatacaataccgtacactgctccgaagacgttctataaccaactgccagactggagcatgcttcttgcagctgtcacaaccattttcctggcagctgagaagcaatggactctgcttgactggaagccgaagaagcctgacatgctggctgacacattcagctttggtaggattatccaagacgggctggtgtttaggcaaaacttcttgattcggtcctacgagattggtgctgatcgtacagcttctatagagacattaatgaatcatttacaggaaacagctctgaaccatgtgaaaactgctggtctcctaggtgatggttttggtgctacgccggagatgagcaaacggaacttaatatgggttgtcagcaaaattcagcttcttgttgagcgatacccatcatggggagatatggtccaagttgacacatgggtagctgctgctggcaaaaatggcatgcgtcgagattggcatgtttgtgactacaactctggtcaaacaatcttgagggctacaagtgtttgggtgatgatgaataagaacactagaagactttcaaaaatgccagatgaagttagagctgaaataggcccgtacttcaatggttgttccgctataacagaggagcagtgtgaaaagttgcctaagccagggaccacatttgatggcactgctaccaaacaattcacaagaaaagggcttactccgaagtggagtgaccttgatgtcaaccagcatgtgaacaatgtgaagtatatcggatggatggctggttctcttgcggcgtctgcattcttccctagcccagggcgaggggaacatgggttttttcccagctga

SEQ ID NO: 48 - protein, 360 aa, Oryza eichingeri
MAGSLAASAFFPSPGSSPAASTRSSKNTTSELPENLSVRGIVAKPNPPPGAMQVKAQAQALPKVNGTKVNLKTTSPEKEDTIPYTAPKTFYNQLPDWSMLLAAVTTIFLAAEKQWTLLDWKPKKPDMLADTFSFGRIIQDGLVFRQNFLIRSYEIGADRTASIETLMNHLQETALNHVKTAGLLGDGFGATPEMSKRNLIWVVSKIQLLVERYPSWGDMVQVDTWVAAAGKNGMRRDWHVCDYNSGQTILRATSVWVMMNKNTRRLSKMPDEVRAEIGPYFNGCSAITEEQCEKLPKPGTTFDGTATKQFTRKGLTPKWSDLDVNQHVNNVKYIGWMAGSLAASAFFPSPGRGEHGFFPS

SEQ ID NO: 49 - DNA, 1284 bp, Oryza eichingeri
atggctggttctcttgcagcgtctgcattcttccctggcccagggtcttcccctgcagcatcagctagaagttctaagaacacaaccggtgaattgccagagaatttgagtgtccgcggaatcgttgcgaagcctaatccgcctccgggagccatgcaagtcaaggcgcaggcgcaagcccttcctaaggttaatggaaccaaggttaacctgaagactactagcccagacaaggaggatacaataccatacactgctccgaagacattctataaccaattgccagactggagcatgcttcttgcagctgtcacgaccattttcctggcagctgagaagcaatggactctgcttgactggaagccgaagaagcctgacatgctggctgacacatttggctttggtaggatcatccaagatgggctggtgtttaggcaaaacttcctgattcggtcctacgaaattggtgctgatcgtacagcttctatagagacattaatgaatcatttacaggaaacagcactgaaccatgtgaaaactgctggtctcctaggtgatggttttggtgctacgccggagatgagcaaacggaacttaatatgggttgtcagcaaaattcagcttcttgttgagcgatacccatcatggggagatatggtccaagttgacacatgggtagctgctgctggcaaaaatggcatgcgtcgagattggcatgtttgtgactacaactctggtcaaacaatcttgagggctacaagtgtttgggtgatgatgaataagaacactagaagactttcaaaaatgccagatgaagttagagctgaaataggcccgtacttcaatggttgttccgctataacagaggagcagtgtgaaaagttgcctaagccagggaccacatttgatggcactgctaccaaacaattcacaagaaaagggcttactccgaagtggagtgaccttgatgtcaaccagcatgtgaacaatgtgaagtatattggatggatacttgagagtgctccaatttccatactggagaagcacgagcttgcaagcatgaccttggattacaggaaggagtgtggccgtgacagtgtgcttcagtcacttaccaccgtatcaggtgaatgtgtcgatggcaacaaagaatcctccatccagtgtaaccatctgcttcagctggagtccggagcagacattgtgaaggctcacacagagtggcgaccaaagcgagcgcagggcgaggggaacatgggttttttcccagctgagagcgcatga

SEQ ID NO: 50 - protein, 427 aa, Oryza eichingeri
MAGSLAASAFFPGPGSSPAASARSSKNTTGELPENLSVRGIVAKPNPPPGAMQVKAQAQALPKVNGTKVNLKTTSPDKEDTIPYTAPKTFYNQLPDWSMLLAAVTTIFLAAEKQWTLLDWKPKKPDMLADTFGFGRIIQDGLVFRQNFLIRSYEIGADRTASIETLMNHLQETALNHVKTAGLLGDGFGATPEMSKRNLIWVVSKIQLLVERYPSWGDMVQVDTWVAAAGKNGMRRDWHVCDYNSGQTILRATSVWVMMNKNTRRLSKMPDEVRAEIGPYFNGCSAITEEQCEKLPKPGTTFDGTATKQFTRKGLTPKWSDLDVNQHVNNVKYIGWILESAPISILEKHELASMTLDYRKECGRDSVLQSLTTVSGECVDGNKESSIQCNHLLQLESGADIVKAHTEWRPKRAQGEGNMGFFPAESA

SEQ ID NO: 51 - DNA, 1284 bp, Oryza eichingeri
atggctggttctcttgcggcgtctgcattcttccctggcccagggtcttcccctgcagcatcagctagaagttctaagaacacaaccggtgaattgccagagaatttgagtgtccgcggaatcgttgcgaagcctaatccgcctccgggagccatgcaagtcaaggcgcaggcgcaagcccttcctaaggttaatggaaccaaggttaacctgaagactactagcccagacaaggaggatacaataccgtacactgctccgaagacgttctataaccaattgccagactggagcatgcttcttgcagctgtcacgaccattttcctggcagctgagaagcaatggactctgcttgactggaagccgaagaagcctgacatgctggctgacacatttggctttggtaggatcatccaagatgggctggtgtttaggcaaaacttcctgattcggtcctacgaaattggtgctgatcgtacagcttctatagagacattaatgaatcatttacaggaaacagcactgaaccatgtgaaaactgctggtctcctaggtgatggttttggtgctacaccggagatgagcaaacggaacttaatatgggttgtcagcaaaattcagcttcttgttgagcgatacccatcatggggagatatggtccaagttgacacgtgggtagctgctgctggcaaaaatggcatgcgtcgagattggcatgtacgggactacaactctggtcaaacaatcttgagggctacaagtgtttgggtgatgatgaataagaacactagaagactttcaaaaatgccagatgaagttagagctgaaataggcccgtacttcaatggtcgttctgttatcacagaggagcagggtgaaaagttgcctaagccagggaccacatttgatggcgctgctaccaaacaattcacaagaaaagggcttactccaaagtggagtgaccttgatgtcaaccagcatgtgaacaatgtgaagtatattggatggatacttgagagtgctccaatttcgatactggagaagcacgagcttgcaagcatgaccttggattacaggaaggagtgtggccgtgacagtgtgcttcagtcacttaccaccgtatcaggtgaatgtgtcgatggcaacaaagaatcctccatccagtgtaaccatctgcttcagctggagtccggagcagacattgtgaaggctcacacagagtggcgaccaaagcgagcgcagggcgaggggaacatgggttttttcccagctgagagcgcatga

SEQ ID NO: 52 - protein, 427 aa, Oryza eichingeri
MAGSLAASAFFPGPGSSPAASARSSKNTTGELPENLSVRGIVAKPNPPPGAMQVKAQAQALPKVNGTKVNLKTTSPDKEDTIPYTAPKTFYNQLPDWSMLLAAVTTIFLAAEKQWTLLDWKPKKPDMLADTFGFGRIIQDGLVFRQNFLIRSYEIGADRTASIETLMNHLQETALNHVKTAGLLGDGFGATPEMSKRNLIWVVSKIQLLVERYPSWGDMVQVDTWVAAAGKNGMRRDWHVRDYNSGQTILRATSVWVMMNKNTRRLSKMPDEVRAEIGPYFNGRSVITEEQGEKLPKPGTTFDGAATKQFTRKGLTPKWSDLDVNQHVNNVKYIGWILESAPISILEKHELASMTLDYRKECGRDSVLQSLTTVSGECVDGNKESSIQCNHLLQLESGADIVKAHTEWRPKRAQGEGNMGFFPAESA

SEQ ID NO: 53 - DNA, 1660 bp, Oryza sativa
gtacatgtaggtcttgtttagatcccaaaaaattttagccaaaacctcacatcaaatatttggacacatgcacccctaccagtgtgtggaggcattgcatacacgaaacatggaaaaggaatcaacttgagaggttagacctgctagctctactaggtctggatggtcatgcatttttttttgaaaaaaaccacgctgcaagctcgacagcctcaacctcaatggcaaccatgacaataatatgcatgacaatggtgtaggagaaaagacacgtcgataaccaaagggcgcggctgcgcatacaaaggcggagagaaggaacgatggtggctcaaaaagaaagagcgtcggtggcagtggtgcgtggagcgacactaaagttagtggttgctgatggtctcacacaatccctaatcgaaatatttattttttttcacttagtattgctgatccgtgggccaccagccaatcataaagaaaaatgttgagataaaaggtggagtatcttccccttccttccctttttgactcgaaaaaaaaaagcgtcggtggcggccgtgcgtgtaacaacactaaagttagtggttgctggtggtctgacacaatccctaatcaagtttgataataataataatttatttcctcttattagtattgctgatgcgtgggccaccaatcaatcgtaaagaaaaaaaatgttgagataaaaggtgggggtatcttctccttctctttttttttggctaaaataaaagtggtttctggtagtctgacacaatctctaatcgaaatatttatttttttctcttagtattgctgatacgtgggccaccagccaataataaagaaaaaaaatgttagagataaaaggcggagagtatcttccccttccttttttttggcgtaaatgaaagaaaagagaaaatctcccgtcgtctccttccttgcgccaagaaagacgagccgcggctcaacaccggaggggaggggcgccgatctccatcgccaaggagagcagagcaggggaggggatcctggtgagcctcctcttcctgattcatctctctcccattctagcttcgggggactacttttgcctggaatttgctcgcgttcgttcgtgcgttcgttcgttaaccctagcttcttctcttctagatctggaggaagctcttctcctccttaatttcagagccttaatacaagtagtaacagtttaacctcccccatgtcccaagttggatccgcccctgcgagttccgatattgggtcctcccaattctcaatgccattttgttcatcggggggcatatggttcatttttgcctgcattgattcaaatgtggtttcgaatcgtttgtgaaattcgcgggtgtacttgtttatgatacatgaggccttttttcccccatgaggaggcaaactttttagtgggtggatccactagttcatgcctcaattttttttctcctcttttaagttttccaaagagctacattgttgtaaagtgtctgatacaattgattgtttattcaggttagcgcttttggcgtgtgattgatttctaaacgaattttgggccgtgaggggaagttcaatcatggcagggtctcttgccgcctcagcattcttcccaggtccaggctcatctcctgcagca

SEQ ID NO: 54 - DNA, 1541 bp, Oryza sativa
acagaaatttcgctggccatgcacataatcttctctttgtcaaagagctggaatccaaaatgattgctcgaagatttcgtgaagatagatagaaccatcggctagcaaaggagaggaaataaaaaaacaaaaaaaaagtttttttgtgggctccaccttgcgctgcactgagctgaccaaattgaccataccgcacagagactgagggaggggcacttccgtcatttcgtataagcgtatacgaatacgtatctcatacgcgctctgtatatatagacggtaacggctccgcgtcgtgtgagttggcgagcccgaggagcggaggcggccacaagtctaatccgcgtcgtctgcgcgttcgtgggcgaggaggagaaagaagaggaggaagagagggaagggggcttgatttgatttgggcgcgtctcgtggagtatccggtgagttcttggcgatctggcgaggcgagtgatgagtgattcctgctgctgctgggggattttggcgtgattttcgttggttgcattttgtttctttttttttgtatcgatttgttggagctttattcggtagatctggtcgattccatggtgagttgtatcggcgccggagtgatagctgattctgtttttttgtgtgattttttttttgttttggaaatagggtttgtgtcgaattgagggcattttttttccttaggcaatgcaggatttcgttttgtatgtttttgcgtggaatggatatgaacagacctcgaacaaatggaagaatttgtattttgtatgatggattgcaatgcgatacttgttttggggcgtgattcgattgaaataaatgaaatattagagttattttgggattcctgtttgctgcgcctttttttttagcatttcttgatatgaacaagagaagaagggctgaatttttttcttagctttggaggcatttactgtcccagtattttctcctaccggaagcagaatattttgtttgattggagggttgcctccctttgccaaattgaatcaaatgttctcggatgttttaaaatttccgtggactctttttgccccaggggagaccgcttttagcagctggatcccgtgttttcatttcaagttcttgttttcctagtctccatatatttctgattgttaactcgtattctctacctcacatatgcaaaatcacacttgcgtcgttctgtaattagttagattctgcaagaaaaatccggaattttcaagcatgctagtagttttaaattgatgccatgttttttagacaatgttaattgatgccatatgactataggacacattatattgcgtttctgaatataccacctcatgaaactcataattttgttgattaattgttcaggttgccccttctagtgtgtaacttggagcaaatttggaccctgagacgcaaatcagtcatggctggttctcttgcggcgtctgcattcttccctgtcccagggtcttcccctgcagcttcggctagaagctctaagaacacaaccggtgaattgccag

SEQ ID NO: 55 - DNA, 1560 bp, Oryza sativa
ttctcgtatcctagcccatatatttttacagattcgggttcaagctcgcataatatcgggtatccaattttctctggcatattcttcatgcaagccttgagttgagtgaacgatcgggaaatacctgccccattttcacccctaccagtgggtgggggcattgcatgaacgaaacatggaaaaggaatcgacttgagaggctagacctgctagctctactgggtctggatggtcatgcatttatttgaaaaaaaaacacgccgcaagctcgataaccccgacctcaacggcaaccatgacaacaatatgcatgacattgggggaggagaaaagacatgttgataactagagggcgcggctacgcatgtaaatgcggagagaaggaacgacggtagctcaaaaagtaagagcgtcggtggcggtggtgcgtggagcgacactaaagttagtggttgctggtggtctgacacaaatccctaatcgaaatatttattttttctcttagtattactgatacgtgggccacccgccaattataaagaaaaatgttgagataaaaggtggagtatctttcccttccttcccttttttgccttaaaaaaaagagcgtcggtgacggccgtgcgtgtagcaatactaaagttagtggttgatggtggtctgacacaatccctaatcgagtttgataataatatttatttttctcttagtatcgctgatacgtgggccaccagccaatcataaaggaaaaaaaatgttgagataaaaggtggatagtatcttcccccttccttcccttttttggcgtaaaagaaagaggagaaattctcccgtcgtctccttccttgcgccaagaaagacgagccgcggctcaacagcggagtggaggggcgccgatctccatcgccgaggagagcagagcaggggaggggaggggatcctggtgagcctcctcttcctgattcacctctctctcattctagcttcgggggactacttttgcctcgaatttgcttgcgttcgttcgttaaccctagcttcttctcttctagatctggaggaagctcttctcctccttaatttcagagccttaatacaagtagtaacagtttaaccccccccccccccatgtcccaagttggatccgcccctgcgagttccgatattgggtcctcccaattctcaatgccattttgttcatcggggggcatatggttcattttgcctgcattgattcaaatgtggtttcgaatcgtttgggaaattcgcgggtgtacttgtttatgatatatgaggccttttttttccccatgaggaggcaaactttttagtgggtggatccactagttcatgcctcaattttttttctcctcttttaagttttccaaagagctacattgttgtaaagtgtctaatacaattgattgtttattcaggttagcgcttttggcgtgtgattgatttctaaacgaattttgggccgtgaagggaagttcaatcatggcagggtctcttgccgcctcagcattcttcccaggtccaggctcatctcctgcagca

SEQ ID NO: 56 - DNA, 1678 bp, Oryza eichingeri
acagaaatttcgctggccatgcacataatcttctctttgtcaaagagctggaatccaaaataattgctcgaagatttcgtgtagatagaaccatcggccagcaaaggagtggaaaaagaaaacgtttttttgtgggccccaccggcgctgcactgagctgaccaaatgactataccgcacagagggaggggggcatttccgtcctttcgtatagacgtatatgaatacgtatctcatacgcgctctgtgtatatagacgcacctgcgccagaggagacggtaacggctcagcgaaggggagagagagaagaaggaaaaaaaaactcatctctctctctctctcttgtttctctctgcctcgcgtcgtgtgagttggcgagcccgaggagcggaggccacaagtctaatccgccgtatctaatccgctcgaccgcgtctgcgcgtgcgtgggtgaggagaaagaggaggaggtggaggagaaagagagggggcttgatctgggcgcttctcgtggagtatccggtgagttcttggcgatctggcgaggcgagtggtgagtggctccgcgtgtgctgctgccgggggattttggcgtgattttcgttggttgcattttgttttttttgtgtatcgatttgttggagcttattcggtagatctggtcgattacatggtgagttgtataggcgccagagtgatagctgattttgttttggtgtaaattttgttttggaaggagggtttgtgtcgatttgagggcatttttcctcgggcaatgcaggatttggatttgtatgtttttgcatggaatggatatgaacggacctcaaacaaatggaggagtttgtactttggatggattgcaatgtggttttgaggcgtgattcggttgaagaaatgaactaaggaatattcgagttattttgggattcctgtttgctgcgcctttttttagcatttcttgatatgaacaagagaaaaagggctgattttttccttagctttggaggcatttactgtcccagtattttctcctaccggaagcagaatattttgtttgattggagggttgtctccttttgccaaatcgaatcaaatgctctcggatgttttgaaatttcggtggactccttttgcccaagggaggccacttttagcagctgtggatcccgtgttttcattcaagttcttgttttcctagtctccatatatttctgattattaactcggattctctacatcaaatatgcgaaatcacacttgcgtcgttctgtagttagttaggttctgcaagacaaatccgaaatttttaagcatgctgtcatagtatcattggattcccccttttactgggaagaaagttctaccttttgtgctttcggtagtttttaattgatgccatgttttttagataatgttaattgatgccatgtgactataggacacattatattgcgtttctgaatatatcacctcatgaaactcataattttgttgattatttgttcaggttgccccttctagcgtgtagcttcgagcaaatttggactctgaggcgcatttcggtcatggctggttctcttgcagcgtctgcattcttccctagcccagggtcttcccctgcagcatcgactagaagttctaagaatacaaccagtgaattgccagagaatttgag

SEQ ID NO: 57 - DNA, 1307 bp, Oryza eichingeri
  misc_feature (462)..(462): n is a, c, g, or t
acagaaatttcgctggccatgcacaatcttctctttgtcaaagagctggaatccaaaatgattgctcgaagatttcgtgtagatagatagaaccatcggccagcaaaggagaggggaacaaaaaggaaaaaagtctttttgtgggccccacctgcactgcactgggttgaccaaattgaccataccgctcagaggggggggggcatttccgtcctttcgtataaacgtatacgaatacgtatctcacacgcgctctgtatatatagacggtaacggctccgcgaaggagagagaagaagaagaaaaaaaaagtcatctttctctctcttgtttctctctgcctcgagtcgcggctgaacaggggaggggcggcgatctccatctggcgagcagagcagggaaggggaggggatcctggtgagcatccacatcctttttctgattcatatatctctcccaccngggagtacttttgtctggaatttgcttgcattaaccctagcttctcttgtagatctggaagaagctcttctcttaatttcagagccttaaccttaatacaagtaacagtttgttgtttgttcccccaaaagtttgctgcgcgtttttttggcatctcttgatatgaacaagagaaacaagctgaattttttcttacctttggaagcatttaccgtcccagtattttctcctaccggtagtagaatattttgtttgattggaggcttgccttcttttgctaaatcgaatcaaatgctctcggatgtttttaaaatttcggtggactccttttgccccaagggaggccagttttagcagctggatcccgtgttttcatttcaacttcttgttttccttgtctccatatatttctgattgttaactcggattctctacctcaaatatgtaatatcacactttaagacaaatccggaattttaagcatgctatcatagtatcattagattcccccttttacagggaagaaaagttctacattttgtgctttcggtagcttttaattgatgccatgttttttagacaatgttaattgatgccatgtgactatagggcacattatattgcgtttctgaatatatcacctcatgaaactgataattttgttgattatttgttcagtttgcccttctagtgtgtaacttcgagcaaatttggaccctgaggcgcagttcagtcatggctggttctcttgcagcgtctgcattcttccctggcccagggtcttcccctgcagcatcagctagaagttctaagaacacaaccggtgaattgccagagaatttgag

SEQ ID NO: 58 - DNA, 398 bp, Oryza sativa
atttccttgctaggtgagacttgagtggtgctagtctggctgcaaatttatagaagtatgtgaaaatttgaggtcagaatacaagtaattgaatggaccaatctaatgagttctgtagctttagaataattaatgttaacataaaaatatgttcatgaaatcaggtccttctgcattttgttgttaaccgaattccacattcttctttagttctcacaagtacagacaagtatcttgtaatggtggattcttttttggaaaacaaacttcattacatattttgtgtgatccatctatgccttgtgcccttgttacctttttttccctacaccttgttttctcttgtacttagttttgcattgtataaccttttgctgtactcgtgtcttgtactgtag

SEQ ID NO: 59 - DNA, 605 bp, Oryza sativa
gtatggatcctttctttgagtgattacctggtatcgtgtaattcttcatttgtgtatactgtatttgagagtttgaaaaaatttccatagaaaataataacatttgttgtttacaaatggtcccgccaaaacagtggaatttatattggggatgtacataaaaggagtgtaaagttctaatgtgcttatgctaacttcctttccatgatctaaagttgttaccttacggtatgctatttattggatctatattgcattttacttggtaaatctatctgaggttccagcttttgatatttaagttttcctatgtttaattcaaaatattctcacgtgaatcgcaaacctcaccaggagtacaataaattcgttttattattattgtaggctgtgttatttctagtccatggttcggtgtcttgaaatttcagtgccaaaattgggatggatctggttacatcttcaagtctaataaatgatcacaccgactttattgtgtgatttgattatagcagggtcttgcaacataaatacaagctattaattgtgaaaggagaaatgagatctttggtgagatcatgagaatagggtataacagacacaat

SEQ ID NO: 60 - DNA, 1384 bp, Oryza sativa
gtatggatcctttctttgagtgattacctggtatcgtgtaattcttcatttgtgtatactgtatttgagagtttgaaaaaatttccatagaaaataataacatttgttgtttacaaatggtcccgccaaaacagtggaatttatattggggatgtacataaaaggagtgtaaagttctaatgtgcttatgctaacttcctttccatgatctaaagttgttaccttacggtatgctatttattggatctatattgcattttacttggtaaatctatctgaggttccagcttttgatatttaagttttcctatgtttaattcaaaatattctcacgtgaatcgcaaacctcaccaggagtacaataaattcgttttattattattgtaggctgtgttatttctagtccatggttcggtgtcttgaaatttcagtgccaaaattgggatggatctggttacatcttcaagtctaataaatgatcacaccgactttattgtgtgatttgattatagcagggtcttgcaacataaatacaagctattaattgtgaaaggagaaatgagatctttggtgagatcatgagaatagggtataacagacacaatatttccttgctaggtgagacttgagtggtgctagtctggctgcaaatttatagaagtatgtgaaaatttgaggtcagaatacaagtaattgaatggaccaatctaatgagttctgtagctttagaataattaatgttaacataaaaatatgttcatgaaatcaggtccttctgcattttgttgttaaccgaattccacattcttctttagttctcacaagtacagacaagtatcttgtaatggtggattcttttttggaaaacaaacttcattacatattttgtgtgatccatctatgccttgtgcccttgttacctttttttccctacaccttgttttctcttgtacttagttttgcattgtataaccttttgctgtactcgtgtcttgtactgtaggcttctgctatcaatgatcccaaaaagcatgaaacttctatgaaaaatgaaagcctgaatactgccctgtcatctgacgatatgatgatcgacaatatacctctatgttctcgtgagtcaactctcgcagtcaatatttcaagtgccccgagccaactggttggaatggttggtttaactgacagctcacctgctgaagttggtacatctgagttgcatcagatgaatagctctggaaatgctatgcaggagtcacagcctgaaagtgtggctgaaaagtctgcagaggatggttataactggcgcaaatatgggcaaaagcatgttaagggaagtgagaacccgagaagctattacaagtgcacacatcctaactgtgat

SEQ ID NO: 61 - DNA, 64 bp, Artificial Sequence
  Consensus CT-rich motif
aaggagagagaagaagaagaaaaaaaaactcatctttctctctcttgtttctctctgcctcgag

SEQ ID NO: 62 - DNA, 36 bp, Cauliflower mosaic virus
accaatctctctctacaaatctatctctctctataa

SEQ ID NO: 63 - DNA, 64 bp, Oryza eichingeri
aaggagagagaagaagaagaaaaaaaaagtcatctttctctctcttgtttctctctgcctcgag

SEQ ID NO: 64 - DNA, 68 bp, Oryza brachyantha
aaggagagagaagaagaagaagaagaaaaaaactcatctttctctctcttgtttctctctgcctcgag

SEQ ID NO: 65 - DNA, 64 bp, Oryza latifolia
aaggagagagaagaagaagaaaaaaaaactcatctttctctctcttgtttctctctgcctcgac

SEQ ID NO: 66 - DNA, 1199 bp, Oryza brachyantha
acagaaatttcgctggccatgcacaatcttctctttgtcaaggagctggaatccaaaatgattgctcgaagatttcgtgtagatagatagaaccatcggccagcaaaggagaggggaaaaaaaaaatgaaaaacgtctttttgtgggccccacctgcactgcactgagttgaccaagttgaccataccgctcagagggggggcatttccgtcctttcgtataaacgtatacgaatacgtatctcacacgcgctctgtatatatagacggtaacggctccgcgaaggagagagaagaagaagaagaagaaaaaaactcatctttctctctcttgtttctctctgcctcgagtcgcggctgaacaggggaggggcggcgatctccatctggcgagcagagcagggaaggggaggggatcctggtgagcatccacatcctttttctgattcatatctctctcccaccgggagtacttttgtctggaatttgcttgcattaaccctagcttctcttgtagatctggaagaagctcttctcttaatttcagagccttaaccttaatacaagtaacagtttgttgtttgttcccccaaaagtttgctgcgcgtttttttagcatctcttgatatgaacaagaggaacaagctgaattttttcttagctttggaagcatttaccgtcccagtattttctcctaccggtagtagaatattttgtttgattggagggttgccttcttttgctaaattgaatcaaatgctctcggatgttttttaaaatttcggtggactccttttgccccaagggaggccagttttagcagctggatcccgtgttttcatttcaacttcttgttttccttgtctccatatatttctgattgttaactcggattctctacctcaaatatgtaatatcacacttaaagacaaatccggaattttaagcatgctatcatagtatcattagattccccctttacagggaagaaaagttctacattttgtgctttcggtagcttttaattgatgccatgttttttagacaatgttaattgatgccatgtgactataaggcacattatattgcgtttctgaatatatcacctcatgaaactgataattttgttgattatttgttcagtttgcccttctagtgtgtaacttcgagcaaatttggaccctgaggcgcagttcagtc

SEQ ID NO: 67 - DNA, 1189 bp, Oryza latifolia
acagaaatttcgctggccatgcacaatcttctctttgtcaaagagctggaatccaaaatgattgctcgaagatttcgtgtagatagatagaaccatcggccagcaaaggagaggggaacaaaaaggaaaaaagtctttttgtgggccccacctgcactgcactgagttgaccaaattgaccataccgctcagaggggggcatttccgtcctttcgtataaacgtatacgaatacgtatctcacacgcgctctgtatatatagacggtaacggctccgcgaaggagagagaagaagaagaaaaaaaaactcatctttctctctcttgtttctctctgcctcgactcgcggctgaacaggggaggggcggcgatctccatctggcgagcagagcagggaaggggaggggatcctggtgagcatccacatcctttttctgattcatatatctctcccaccgggagtacttttgtctggaatttgcttgcgttaaccctagcttctcttgtagatctggaagaagctcttctcctaatttcagagccttaaccttaatacaagtaacagtttgttgtttgttcccccaaaagtttgctgcgcgtttttttggcatctcttgatatgaacaagagaaacaagctgaattttttcttagctttggaagcatttaccgtcccagtattttctcctaccggtagaatattttgtttgattggaggcttgccttcttttgctaaatcgaatcaaatgctctcggatgtttttaaaatttcggtggactccctttgccccaagggaggccagttttagcagctggatcccgtgttttcatttcaacttcttgttttccttgtctccatatatttctgattgttaactcggattctctacctcaaatatgtaatatcacactttaagacaaatccggaattttaagcatgctatcatagtatcattagattcccccttttacagggaagaaaagttctacattttgtgctttcggtagcttttaattgatgccatgttttttagacaatgttaattgatgccatgtgactatagggcacattatattgcgattctgaatatatcacctcatgaaactgataattttgttgattatttgttcagtttgcccttctagtgtgtaacttcgagcaaatttggaccctgaggcgcagttcagtc

SEQ ID NO: 68 - DNA, 1193 bp, Artificial Sequence
  Consensus FatB6 promoter
acagaaatttcgctggccatgcacaatcttctctttgtcaaagagctggaatccaaaatgattgctcgaagatttcgtgtagatagatagaaccatcggccagcaaaggagaggggaacaaaaaggaaaaaagtctttttgtgggccccacctgcactgcactgagttgaccaaattgaccataccgctcagagggggggcatttccgtcctttcgtataaacgtatacgaatacgtatctcacacgcgctctgtatatatagacggtaacggctccgcgaaggagagagaagaagaagaaaaaaaaactcatctttctctctcttgtttctctctgcctcgagtcgcggctgaacaggggaggggcggcgatctccatctggcgagcagagcagggaaggggaggggatcctggtgagcatccacatcctttttctgattcatatatctctcccaccgggagtacttttgtctggaatttgcttgcgttaaccctagcttctcttgtagatctggaagaagctcttctcttaatttcagagccttaaccttaatacaagtaacagtttgttgtttgttcccccaaaagtttgctgcgcgtttttttggcatctcttgatatgaacaagagaaacaagctgaattttttcttagctttggaagcatttaccgtcccagtattttctcctaccggtagtagaatattttgtttgattggaggcttgccttcttttgctaaatcgaatcaaatgctctcggatgtttttaaaatttcggtggactccttttgccccaagggaggccagttttagcagctggatcccgtgttttcatttcaacttcttgttttccttgtctccatatatttctgattgttaactcggattctctacctcaaatatgtaatatcacactttaagacaaatccggaattttaagcatgctatcatagtatcattagattcccccttttacagggaagaaaagttctacattttgtgctttcggtagcttttaattgatgccatgttttttagacaatgttaattgatgccatgtgactatagggcacattatattgcgtttctgaatatatcacctcatgaaactgataattttgttgattatttgttcagtttgcccttctagtgtgtaacttcgagcaaatttggaccctgaggcgcagttcagtc

SEQ ID NO: 69 - DNA, 942 bp, Artificial Sequence
  Consensus FatB6 promoter
acagaaatttcgctggccatgcacaatcttctctttgtcaaagagctggaatccaaaatgattgctcgaagatttcgtgagatagatagaaccatcggcagcaaaggagaggaaaaaaaaaaaagttttttgtgggcccacctgcctgcactggtgaccaaattgaccataccgccagaggggggggcattccgtctttcgtataacgtatacgaatacgtatctcaacgcgctctgtatatatagacggtaacggctccgcggggagagaggagaaagtcttctgttgggggaaagggaggggcgggatcatctggcgaggaggagggggattggggcattcttttttgatttaatcttcccccgggtttttttgatttttgtttagtttatgatttcttaatcagtacctaaaagaaatttgttttgttcaaagtttgctgcgctttttttgcattcttgatatgaacaagagaaagctgaattttttcttactttggagcatttacgtcccagtattttctcctaccggagagaatattttgtttgattggaggttgcctctttgcaaatgaatcaaatgtctcggatgttttaaaatttcgtggactcttttgccccagggagccttttagcagctggatcccgtgttttcatttcaattcttgttttcctgtctccatatatttctgattgttaactcgattctctacctcaatatgaaatcacacttgctttattttagattccaagaaaatcattttgctcgtagtttaattgatgccatgttttttagacaatgttaattgatgccattgactataggcacattatattgcgtttctgaatatacacctcatgaaactataattttgttgattattgttcagttgcccttctagtgtgtaacttgagcaaatttggaccctgagcgcatcagtc

(19) United States
(12) Patent Application Publication
(10) Pub. No.: US 2022/0033835 A1
(43) Pub. Date: Feb. 3, 2022

(54) RICE PLANT MATERIAL RESISTANT AGAINST BIOTIC STRESS

(71) Applicant: Chuanxin SUN, Uppsala (SE)

(72) Inventors: Chuanxin SUN, Uppsala (SE); Yunkai JIN, Uppsala (SE)

(21) Appl. No.: 17/276,079

(22) PCT Filed: Sep. 12, 2019
(86) PCT No.: PCT/SE2019/050863

§ 371 Date: Mar. 12, 2021

Related U.S. Application Data
(60) Provisional application No. 62/731,282, filed on Sep. 14, 2018.

(51) Int. Cl. C12N 15/82; C12N 9/16

(52) U.S. Cl. CPC C12N 15/8216; C12N 9/16; C12N 15/8247; C12N 15/8286; C12N 15/8282; C12Y 301/02014

(57) ABSTRACT

A rice plant material having improved resistance against biotic stress factors, including rice brown planthopper and rice blast fungus, is achieved by overexpressing a FatB gene in the rice plant material to cause an increase in oil or triacylglycerol content in the rice plant material.

### TECHNICAL FIELD

It is a general objective to provide a rice plant material having improved resistance against biotic stress, and in particular against rice brown planthopper and rice blast fungus.

### BACKGROUND

Rice is a main staple food in the world and over half of the human population eats rice as a staple food. Yearly production of rice is around 700 million tons. Several problems in rice agriculture related to interactions between rice and the biotic stress factors of insects and microorganisms exist and threaten the human future by an immediate impact on human food security. Those problems include the major insect pest of rice brown planthopper (BPH) (Nilaparvata lugens) and the disease of rice blast fungus (Magnaporthe oryzae), also known as rice rotten neck, rice seedling blight and blast of rice. Annually, the rice brown planthopper and rice blast fungus cause rice yield losses between 12-40% and at the worst even up to 100%. Thus, understanding the interactions between rice and the rice brown planthopper and rice blast fungus is very important for the human food security.

There is therefore a need to provide a rice plant material having improved resistance against biotic stress, and, in particular, against rice brown planthopper and rice blast fungus.

### SUMMARY

The present invention generally relates to a rice plant material having resistance against rice brown planthopper and rice blast fungus.

The present invention is defined in the independent claims. Further embodiments of the invention are defined in the dependent claims.

The rice plant material of the present invention has increased oil (triacylglycerol) content caused by overexpression of a FatB gene, preferably a FatB6 gene. The increased oil or triacylglycerol content caused by overexpression of the FatB gene in the rice plant material improves the resistance of the rice plant material against rice brown planthopper and rice blast fungus.

### BRIEF DESCRIPTION OF THE DRAWINGS

The embodiments, together with further objects and advantages thereof, may best be understood by making reference to the following description taken together with the accompanying drawings, in which:

FIGS. 1A and 1B are images of wild rice (Oryza eichigeri, FIG. 1A) and Nipponbare rice (Oryza sativa L. ssp Japonica, FIG. 1B) in a phytotron.

FIGS. 2A to 2C illustrate identification of high oil, triacylgycerol (TAG), content in leaf sheath and stems of wild rice. FIGS. 2A and 2B indicate high oil content in wild rice (FIG. 2A) compared with Nipponbare rice (FIG. 2B). Scar bar=20 μm. FIG. 2C is a diagram comparing TAG content (% per fresh weight (FW)) in wild rice and Nipponbare. Statistical analysis was performed by one-way ANOVA (**P≤0.01, error bars show standard deviation (s.d.)).

FIG. 3 illustrates gene expression analysis of five key genes in TAG formation in leaf sheath and stems of wild rice and Nipponbare and show relative gene expression levels. Statistical analysis was performed by one-way ANOVA (*P≤0.05 or **P≤0.01, error bars show s.d.).

FIG. 4 illustrates gene expression analysis of Nipponbare FatB2, FatB6 and FatB11 in the tissues of stems and leaf sheath, and seeds.

FIGS. 5A and 5B illustrate oil abundance in the transgenic line (To) of NippFatB6 (FIG. 5A) and control (FIG. 5B). Scar bar=20 μm.

FIGS. 6A to 6C illustrate resistance of the transgenic line (To) of NippFatB6 against rice brown planthopper. FIG. 6A shows triplicates of inoculation of rice brown planthopper on rice plants of NippFatB6 and control (Nipp). FIG. 6B shows the average insect numbers per tiller of the biological triplicates on day 2 after inoculation. FIG. 6C displays an image of more insects on control plants (black arrow) than on NippFatB6 on day 2 after inoculation. Statistical analysis was performed by one-way ANOVA (*P≤0.05, error bars show s.d.).

FIGS. 7A to 7C illustrate resistance of the transgenic line (To) of NippFatB6 against rice blast fungus.

FIG. 7A displays an image of lesion size on day 5 after inoculation of rice blast fungus. FIGS. 7B and 7C indicate the lesion width (FIG. 7B) and length (FIG. 7C) on day 5 after inoculation respectively. Statistical analysis was performed by one-way ANOVA (*P≤0.05 or **P≤0.01, error bars show s.d.).

FIGS. 8A and 8B illustrate the sugar-sensing competitive transcription factor binding system controlling the coordinated starch and fructan synthesis in barley. The sugar-responsive activator-repressor SUSIBA2-SUSIBA1 transcription factor duo orchestrates the coordinated starch and fructan in barley via sucrose/glucose/fructose (Suc/Glc/Fru) signaling. When sugar level is low (FIG. 8A), a recruited transcription factor or complex (a ball with a question mark) binds to the sugar-responsive sequence in the SUSIBA1 promoter and activates SUSIBA1 expression. High expression of SUSIBA1 results in a high level of SUSIBA1 that binds to the W-box in the SUSIBA2 promoter preventing SUSIBA2 binding, and to the cis elements in fructan gene promoters, and represses expression of SUSIBA2 and fructan genes, and as a consequence, low synthesis and content of starch and fructan at a low sugar level. Upon increasing of sugar to a high level (FIG. 8B), the level of the transcription factor/complex decreases and eventually goes to zero when sugar continues to increase. Without binding of the transcription factor or complex to the sugar-responsive sequence in the SUSIBA1 promoter, expression of SUSIBA1 is low. The low expression of SUSIBA1 leads to high expression of fructan genes and a progressive increase of SUSIBA2 expression. SUSIBA2 binds to the W-box in its own promoter and enhances its own expression. More SUSIBA2 binds to the W-box and more SUSIBA2 transcripts are produced. Such positive autoregulation will lead to high expression of SUSIBA2 and high synthesis of starch. Thus, at a high sugar level, high synthesis and content of starch and fructan are generated.

FIG. 9 illustrates an alignment of FatB6 promoter sequences of three wild rice with Nipponbare. Jinsui (Oryza eichingen) (corresponding to nucleotides 1-1,235 in SEQ ID NO: 57), Duanhua (Oryza brachyantha) (SEQ ID NO: 66), and CCDD (Oryza latifolia) (SEQ ID NO: 67) are aligned with Nipponbare (corresponding to nucleotides 1-1,367 in SEQ ID NO: 54) by DNASTAR lasergene 14. Nucleotide sequences with CT-rich motifs similar to the 35S promoter CT-rich motifs (Pauli et al 2004) are boxed.

FIG. 10 illustrates relative gene expression level of FatB6 in three wild rice compared with Nipponbare, indicating a role of the CT-rich motifs in the FatB6 promoters. Statistical analysis was performed by one-way ANOVA (*P≤0.05, error bars show s.d.).

### DETAILED DESCRIPTION

It is a general objective to provide a rice plant material having improved resistance against biotic stress, and in particular against rice brown planthopper and rice blast fungus.

Wild rice, such as Oryza eichigeri, O. brachyantha and O. latifolia, generally has higher resistance against biotic stress factors of insects and microorganisms as compared to cultivated rice (Asian rice, Oryza sativa, and African rice, Oryza glaberrima). In particular, wild rice is more resistant against the major insect pest of rice brown planthopper (BPH) (Nilaparvata lugens) and the disease of rice blast fungus (Magnaporthe oryzae). As is shown herein, the higher resistance against such biotic stress factors is at least partly dependent on high oil, triacylglycerol (TAG), content in the leaves, leaf sheath and stems in wild rice as compared to cultivated rice. Experimental data as shown herein indicates that the higher oil or TAG content in wild rice is mainly associated with significantly increased expression of FatB genes, in particular the FatB6 gene, in wild rice as compared to cultivated rice. The high expression of FatB genes, in particular the FatB6 gene, in wild rice is due to the wild rice-specific promoter, which has been modified in cultivated rice during rice evaluation and domestication. For instance, the wild rice FatB6 promoter comprises a CT-rich motif that is lacking in the cultivated rice FatB6 promoter. Increasing expression of FatB genes, in particular the FatB6 gene, in cultivated rice led to increase in oil or TAG content and improved resistance against rice brown planthopper and rice blast fungust.

A FatB gene encodes an enzyme acyl-acyl carrier protein (ACP) thioesterase B (FatB or FATB), EC 3.1.2.14. Cultivated rice of variety Nipponbare (Oryza sativa L. ssp. Japonica) contained three FatB genes located on chromosomes 2, 6 and 11 and are denoted FatB2, FatB6 and FatB11, see SEQ ID NO: 41 to 46. Wild rice also comprises three corresponding FatB genes, see SEQ ID NO: 47 to 52. The expression of the three FatB genes were significantly higher in wild rice as compared to cultivated rice. This difference in gene expression of FatB genes seems to be the cause of higher oil and TAG content in wild rice as compared to cultivated rice and thereby the cause of the higher resistance of wild rice against biotic stresses, such as rice brown planthopper and rice blast fungus, as compared to cultivated rice.

The genus Oryza consists of more than 20 species, including about 20 wild Oryza species and two cultivated species (O. sativa and O. glaberrima).

An embodiment relates to a rice plant material having higher oil or TAG content as compared to a wild-type rice plant material, and in particular a higher oil or TAG content in leaves, leaf sheath and/or stems.

An embodiment relates to a rice plant material characterized by overexpression of a FatB gene.

An embodiment relates to a rice plant material comprising a FatB gene adapted for overexpression of a FatB enzyme.

In an embodiment, the FatB enzyme is selected from the group consisting of FatB2 as defined in SEQ ID NO: 42 or 48, FatB6 as defined in SEQ ID NO: 44 or 50, FatB11 as defined in SEQ ID NO: 46 or 52, a FatB enzyme having at least 80% sequence identify with a FatB enzyme as defined in SEQ ID NO: 42, 44, 46, 48, 50 or 52, and a combination thereof. In a particular embodiment, the FatB enzyme has at least 85%, at least 90%, at least 95% or at least 99% sequence identity with a FatB enzyme as defined in SEQ ID NO: 42, 44, 46, 48, 50 or 52. In a particular embodiment, the FatB enzyme having at least 80% sequence identity with a FatB enzyme as defined in SEQ ID NO: 42, 44, 46, 48, 50 or 52 is capable of catalyzing the hydrolysis of the thioester bond that links the acyl chain of acyl-ACP to phosphopantetheine prosthetic group of ACP. Hence, the FatB enzyme has enzymatic activity in hydrolyzing this thioester bond.

In an embodiment, the rice plant material has higher oil and/or TAG content, such as in leaves, leaf sheath and/or stems, as compared to a wild-type rice plant material lacking overexpression of the FatB gene or the FatB enzyme.

The FatB gene is preferably selected from the group consisting of FatB2, FatB6, FatB11 and a combination thereof. Thus, the rice plant material can be characterized by overexpression of the FatB2 gene, overexpression of the FatB6 gene, overexpression of the FatB11 gene, overexpression of the FatB2 and FatB6 genes, overexpression of the FatB2 and FatB11 genes, overexpression of the FatB6 and FatB11 genes, or overexpression of the FatB2, FatB6 and FatB11 genes. In an embodiment, the rice plant material is characterized by overexpression of the FatB6 gene, overexpression of the FatB2 and FatB6 genes, overexpression of the FatB6 and FatB11 genes, or overexpression of the FatB2, FatB6 and FatB11 genes, preferably overexpression of the FatB6 gene.

The FatB gene could be any FatB gene, preferably a plant FatB gene and more preferably an Oryza FatB gene. For instance, the FatB gene could be an O. sativa FatB gene, an O. glaberrima FatB gene, an O. eichigeri FatB gene, an O. brachyantha FatB gene, an O. latifolia FatB gene, or a combination thereof.

The FatB gene could be a heterologous gene or an endogenous gene. For instance, if the rice plant material is an O. sativa plant material, an endogenous FatB gene would be an O. sativa FatB gene, whereas a heterologous FatB gene could be an O. eichigeri FatB gene or an O. glaberrima FatB gene.

Overexpression of the FatB gene can be achieved according to various embodiments. In an embodiment, the native or wild-type promoter of an endogenous FatB gene, or at least a portion thereof, is replaced by another promoter or promoter portion or element, such as enhancement element, that causes an increase in expression of the endogenous FatB gene in the rice plant material. Alternatively, or in addition to replacing the native or wild-type promoter, one or more enhancement elements could be added and operatively linked to the native or wild-type promoter to thereby enhance the activity of the native or wild-type promoter. The another promoter could for instance be a constitutively active promoter or an inducible promoter. Illustrative, but non-limiting, examples of such constitutively active promoters include ARP1, H3F3, HSP, H2BF3 and Cauliflower Mosaic Virus (CaMV) 35S promoter. In an embodiment, the promoter is the barley SBEIIb promoter. Furthermore, if the rice plant material is an O. sativa plant material or an O. glaberrima plant material, the promoter of its endogenous FatB gene can be replaced by a heterologous FatB promoter, such as the corresponding FatB promoter from wild rice, e.g., an O. eichigeri FatB promoter, an O. brachyantha FatB promoter, an O. latifolia FatB promoter, or a combination thereof.

In an embodiment, the heterologous FatB promoter is an O. eichigeri FatB promoter selected from the group consisting of the O. eichigeri FatB2 promoter, the O. eichigeri FatB6 promoter, the O. eichigeri FatB11 promoter, or a combination thereof, preferably the O. eichigeri FatB6 promoter. Corresponding preferred O. brachyantha and O. latifolia FatB promoters include the O. brachyantha FatB6 promoter and the O. latifolia FatB6 promoter.

Experimental data as shown herein indicates that the FatB6 promoter of O. sativa is similar to the corresponding FatB6 promoters of wild rice represented by O. eichingeri, O. brachyantha and O. latifolia except the presence of a CT-rich motif in the wild rice FatB6 promoters that is lacking in the FatB6 promoter of O. sativa. The consensus sequence of this CT-rich motif from O. eichingeri, O. brachyantha and O. latifolia is AAGGAGAGAGAAGAAGAAGAAAAAAAAACTCATCTTTCTCTCTCTTGTTTCTCTCTGCCTCGAG (SEQ ID NO: 61). This CT-rich motif is similar to a corresponding CT-rich motif within a 60-nucleotide region (51) downstream of the transcription start site of the cauliflower mosaic virus 35S RNA, ACCAATCTCTCTCTACAAATCTATCTCTCTCTATAA (SEQ ID NO: 62). The CT-rich motif is involved both in enhancer function and in interaction with plant nuclear proteins (Pauli et al., 2004).

In an embodiment, overexpression of the FatB gene can be achieved by the introduction of one or more CT-rich motifs into the FatB promoter, preferably in an O. sativa FatB promoter or in an O. glaberrima FatB promoter. In an embodiment, the CT-rich motif can be according to the consensus sequence above, according to the CT-rich motif in the O. eichingeri FatB6 promoter AAGGAGAGAGAAGAAGAAGAAAAAAAAAGTCATCTTTCTCTCTCTTGTTTCTCTCTGCCTCGAG (SEQ ID NO: 63), according to the CT-rich motif in the O. brachyantha FatB6 promoter AAGGAGAGAGAAGAAGAAGAAGAAGAAAAAAACTCATCTTTCTCTCTCTTGTTTCTCTCTGCCTCG AG (SEQ ID NO: 64), according to the CT-rich motif in the O. latifolia FatB6 promoter AAGGAGAGAGAAGAAGAAGAAAAAAAAACTCATCTTTCTCTCTCTTGTTTCTCTCTGCCTCGAC (SEQ ID NO: 65), or according to the CT-rich motif in the S1 region of the cauliflower mosaic virus 35S promoter, or a combination thereof.

In another embodiment, overexpression of the FatB gene could be achieved by increasing the copy number of the endogenous FatB gene. Hence, in such an embodiment the rice plant material comprises multiple, i.e., at least two, copies of the endogenous FatB gene. The multiple endogenous FatB genes could all, or at least a portion thereof, be operatively linked to and controlled by a single promoter or different endogenous FatB genes could be operatively linked to and controlled by different promoters, which could be of same promoter type or of different promoter types.

In a further embodiment, overexpression of the FatB gene is achieved by transforming the rice plant material with one or more copies of a heterologous FatB gene, such an O. eichigeri FatB gene, an O. brachyantha FatB gene, an O. latifolia FatB gene, or a combination thereof, if the rice plant material is an O. sativa or O. glaberrima plant material.

Any of the above described embodiments of achieving overexpression of the FatB gene can be combined. For instance, the rice plant material can comprise at least one copy of an endogenous FatB gene and at least one copy of a heterologous FatB gene. In such a case, the different FatB genes can be under control of a same promoter or different promoters.

The rice plant material is not a plant material of wild rice. Hence, the rice plant material is preferably a plant material of cultivated rice. In an embodiment, the rice plant material is an O. sativa plant material or an O. glaberrima plant material.

In a particular embodiment, the rice plant material is an O. sativa plant material or an O. glaberrima plant material having overexpression of a FatB gene.

In an embodiment, the rice plant material is an O. sativa or an O. glaberrima plant material, preferably an O. sativa plant material, comprising a wild rice FatB promoter operatively linked to an endogenous FatB gene. In an embodiment, the wild rice FatB promoter is an O. eichigeri FatB promoter, preferably the O. eichigeri FatB2 promoter, the O. eichigeri FatB6 promoter or the O. eichigeri FatB11 promoter, and more preferably the O. eichigeri FatB6 promoter. Alternatively, or in addition, FatB promoters from O. brachynatha and/or O. latifolia could be used, such as the O. brachynatha FatB6 promoter and/or the O. latifolia FatB6 promoter.

In an embodiment, the endogenous FatB gene is the endogenous FatB2 gene, the endogenous FatB6 gene or the endogenous FatB11 gene, preferably the endogenous FatB6 gene.

In another embodiment, the rice plant material is an O. sativa or an O. glaberrima plant material, preferably an O. sativa plant material, comprising a wild rice FatB promoter operatively linked to a heterologous FatB gene, preferably a wild rice FatB gene. In an embodiment, the wild rice FatB promoter is an O. eichigeri FatB promoter, preferably the O. eichigeri FatB2 promoter, the O. eichigeri FatB6 promoter or the O. eichigeri FatB11 promoter, more preferably the O. eichigeri FatB6 promoter. In an embodiment, the heterologous FatB gene is an O. eichigeri FatB gene, preferably the O. eichigeri FatB2 gene, the O. eichigeri FatB6 gene or the O. eichigeri FatB11 gene, and more preferably the O. eichigeri FatB6 gene. Alternatively, or in addition, an O. brachynatha and/or O. latifolia FatB promoters and/or genes could be used.

For instance, an O. eichigeri FatB promoter could be operatively linked to an O. eichigeri FatB gene, to an O. brachynatha FatB gene and/or an O. latifolia FatB gene; an O. brachynatha FatB promoter could be operatively linked to an O. eichigeri FatB gene, to an O. brachynatha FatB gene and/or an O. latifolia FatB gene; and/or an O. latifolia FatB promoter could be operatively linked to an O. eichigeri FatB gene, to an O. brachynatha FatB gene and/or an O. latifolia FatB gene.

In a further embodiment, the rice plant material is an O. sativa or an O. glaberrima plant material, preferably an O. sativa plant material, comprising a constitutively active or a strong promoter operatively linked to an endogenous FatB gene. In an embodiment, the promoter is the barley SBEIIb promoter. In an embodiment, the endogenous FatB gene is the endogenous FatB2 gene, the endogenous FatB6 gene or the endogenous FatB11 gene, preferably the endogenous FatB6 gene.

Non-limiting examples of rice plant materials include a rice plant, a rice plant cell, rice tissue and rice seed.

Reference to a FatB gene, a FatB enzyme or a FatB promoter herein also encompasses, in an embodiment, a FatB gene, a FatB enzyme or a FatB promoter having at least 80%, preferably at least 85%, at least 90%, at least 95% or at least 99% sequence identity with the referred FatB gene, FatB enzyme or FatB promoter. The FatB gene, FatB enzyme or FatB promoter having at least 80% sequence identity preferably maintains the function of the referred FatB gene, FatB enzyme or FatB promoter, i.e., is capable of encoding a functional FatB enzyme (having acyl-ACP thioesterase activity) in the case of a FatB gene having at least 80% sequence identity, has enzymatic acyl-ACP thioesterase activity in the case of a FatB enzyme having at least 80% sequence identity or is capable of initiating transcription of an operatively linked FatB gene in the case of a FatB promoter having at least 80% sequence identity.

The increase in resistance against rice brown planthopper and rice blast fungus according to the embodiments can advantageously be applied to a rice plant material having a controlled production of carbohydrates, in particular starch. Such rice plant material may also reduce emission of methane, and can thereby be a high-starch and low-methane rice plant material having improved resistance against rice brown planthopper and rice blast fungus. A rice plant material having a controlled production of carbohydrates and a reduced emission of methane that can be used according to the embodiments is disclosed in PCT/SE2018/050335 having publication number WO 2018/182493.

In such a case, the rice plant material also comprises a genomic nucleotide sequence encoding a sugar signaling in barley 2-like transcription factor, referred to as herein SUSIBA2, under transcriptional control of a promoter active in the rice plant material. The genomic nucleotide sequence encoding the SUSIBA2 lacks at least a portion of an activation region of a SUSIBA1 promoter (SUSIBA1 p) present in an intron of a wild-type version of the genomic nucleotide sequence encoding the SUSIBA2 transcription factor.

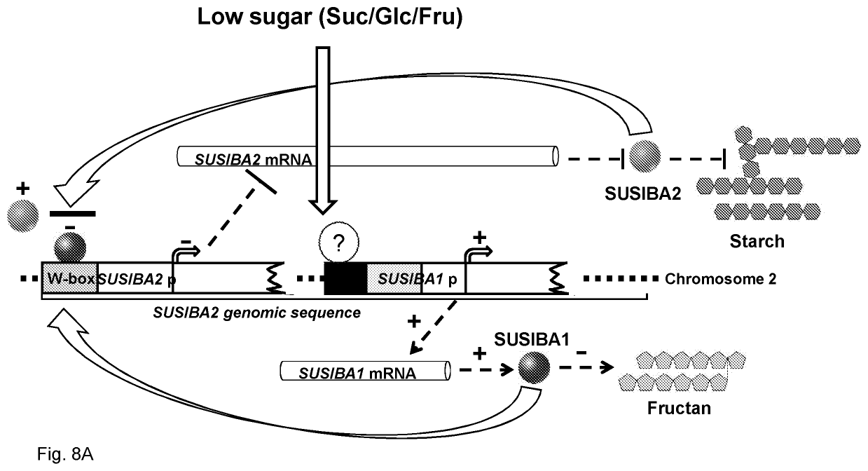

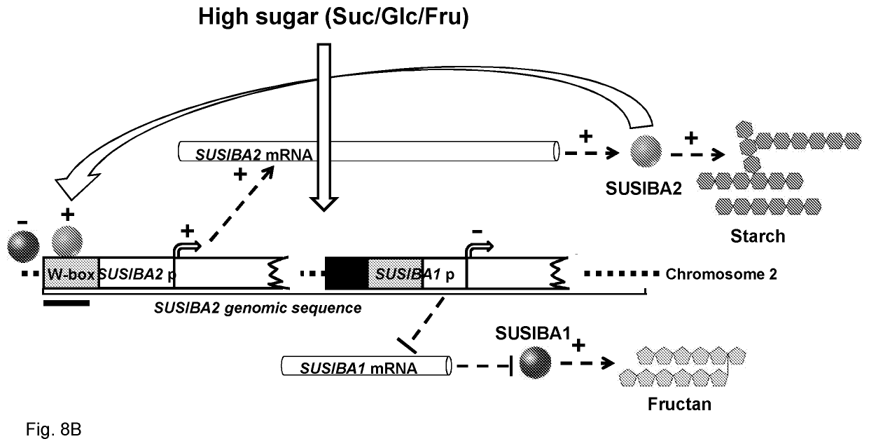

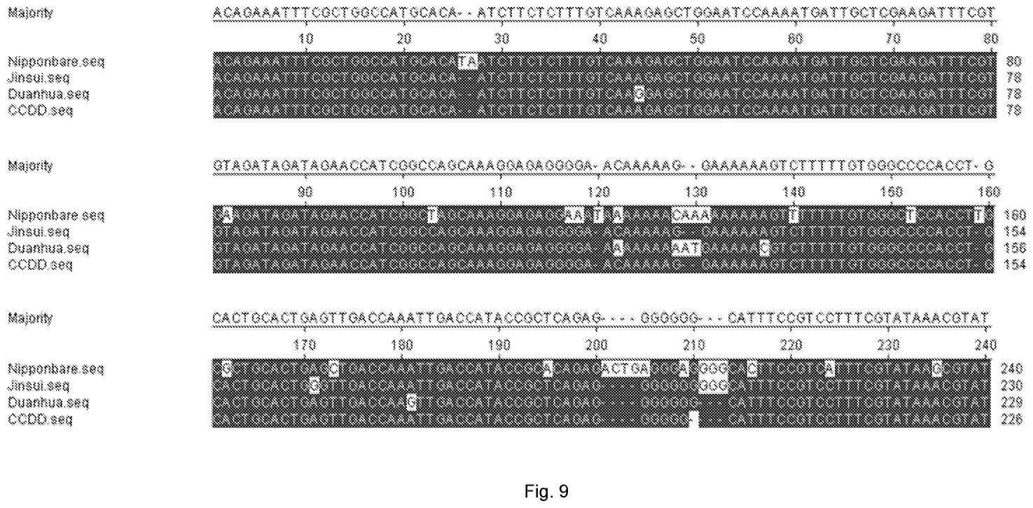

Thus, according to such embodiments, the genomic nucleotide sequence encoding the SUSIBA2 transcription factor, i.e., the SUSIBA2 gene, lacks at least a portion the activation region of the SUSIBA1 p that is otherwise present in an intron in the wild-type version of the SUSIBA2 gene. The absence of at least a portion of the activation region implies that any trans activation factor or complex cannot efficiently bind to the activation region and thereby cannot efficiently activate the SUSIBA1 p. As a consequence, no or only low amount of the SUSIBA1 transcription factor will be produced in the rice plant material regardless of the sugar level in the rice plant material. The absence or low amount of SUSIBA1 transcription factor in the rice plant material in turn implies that the SUSIBA2 transcription factor will outcompete the SUSIBA1 transcription factor for the binding to the SUSIBA2 p, and in more detail to the at least one W-box in the SUSIBA2 p. This will in turn cause activation of the SUSIBA2 p and further production of the SUSIBA2 transcription factor in the rice plant material. The high levels of the SUSIBA2 transcription factor and the low levels of the SUSIBA1 transcription factor in the rice plant material induces production of starch in the rice plant material, see FIGS. 8A and 8B showing the sugar-sensing competitive transcription factor binding system involving SUSIBA1 and SUSIBA2, here exemplified in barley, which, in clear contrast to rice, is capable of synthesizing fructan.

The suppressed expression of the SUSIBA1 gene and thereby low levels of the SUSIBA1 transcription factor, due to the lack or absence of at least a portion of the activation region of the SUSIBA1 p, causes enhanced expression of the SUSIBA2 gene and thereby high levels of the SUSIBA2 transcription factor. The SUSIBA2 transcription factor will in turn activate genes involved in the starch synthesis in the rice plant material.

The rice plant material of these embodiments will thereby be a high-starch rice plant material having improved resistance against rice brown planthopper and rice blast fungus.

The at least a portion of the activation region of the SUSIBA1 p is, in an embodiment, deleted from the wild-type version of the genomic nucleotide sequence encoding the SUSIBA2 transcription factor. As a consequence of this deletion and thereby absence of the at least a portion of the activation region of the SUSIBA1 p, the rice plant material comprises a genomic nucleotide sequence encoding the SUSIBA2 transcription factor and that lacks the at least a portion of the activation region of the SUSIBA1 p. Accordingly, the rice plant material does not comprise any such portion of the activation region of the SUSIBA1 p.

In a particular embodiment, the at least a portion of the activation region of the SUSIBA1 p is deleted by clustered regularly interspaced short palindromic repeat (CRISPR)/CRISPR associated protein 9 (CRISPR/Cas9) mediated deletion from the wild-type version of the genomic sequence encoding the SUSIBA2 transcription factor.

CRISPR/Cas9 is a DNA cutting method that involves expressing the RNA-guided Cas9 endonuclease along with guide RNAs directing it to a particular sequence to be edited. When Cas9 cuts the target sequence, the plant cell repairs the damage by replacing the original sequence with homologous DNA. By introducing an additional template with appropriate homologies, Cas9 can be used to delete, add, or modify genes in an unprecedentedly simple manner. CRISPR/Cas9 is thereby an efficient technology for deleting at least a portion of the activation region of the SUSIBA1 p from the wild-type version of the genomic sequence encoding the SUSIBA2 transcription factor in the rice plant material.

Although CRISPR/Cas9 mediated deletion of at least a portion of the activation region of the SUSIBA1 p is a preferred technology of producing a rice plant material with no or suppressed expression of the SUSIBA1 gene, the embodiments are not limited thereto. Other technologies and techniques known in the art and that can be used to remove or delete genomic nucleotide sequences in rice plant materials can alternatively be used. For instance, promoter deletion could be used to generate or produce a nucleotide sequence encoding the SUSIBA2 transcription factor but lacks at least a portion of the activation region of the SUSIBA1 p that is otherwise present in an intron of the nucleotide sequence (SUSIBA2 gene). The resulting construct can then be agroinfiltrated into the rice plant material.

Agroinfiltration is a method used in plant biology to induce expression of genes in a rice plant material. In the method a suspension of Agrobacterium tumefaciens is introduced into the rice plant material by direct injection or by vacuum infiltration, or brought into association with rice plant material on a support, where after the bacteria transfer the desired produced nucleotide sequence into the rice plant material via transfer of T-DNA.

The first step is to introduce the nucleotide sequence to a strain of Agrobacterium tumefaciens. Subsequently, the strain is grown in a liquid culture and the resulting bacteria are washed and suspended into a suitable buffer solution. For injection, this solution is then placed in a syringe. The tip of the syringe is pressed against the underside of the rice plant material, such as a leaf, while simultaneously applying gentle counter pressure to the other side of the leaf. The Agrobacterium suspension is then injected into the airspaces inside the leaf through stomata, or sometimes through a tiny incision made to the underside of the leaf.

Vacuum infiltration is another way to introduce Agrobacterium deep into rice plant tissue. In this procedure, leaf disks, leaves, or whole rice plants are submerged in a beaker containing the solution, and the beaker is placed in a vacuum chamber. The vacuum is then applied, forcing air out of the intercellular spaces within the leaves via the stomata. When the vacuum is released, the pressure difference forces the Agrobacterium suspension into the leaves through the stomata into the mesophyll tissue. This can result in nearly all of the rice cells in any given leaf being in contact with the bacteria. Once inside the rice plant material the Agrobacterium remains in the intercellular space and transfers the nucleotide sequence as part of the Ti plasmid-derived T-DNA in high copy numbers into the rice cells.

In an embodiment, the genomic nucleotide sequence encoding the SUSIBA2 transcription factor is a genomic endogenous nucleotide sequence. In a particular embodiment, the genomic endogenous nucleotide sequence is present in a chromosome of the rice plant material. Thus, at least a portion of the activation region of the SUSIBA1 p has, according to the embodiments, been deleted, such as by CRISPR/Cas9-mediated deletion, from the genomic endogenous nucleotide sequence, preferably present in a chromosome of the rice plant material.

In an embodiment, a portion of the activation region of the SUSIBA1 p is deleted from the nucleotide sequence encoding the SUSIBA2 transcription factor. In such a case, the deleted portion is preferably selected to correspond to the sub-region or sequence of the activation region to which the trans activation factor or complex binds. Accordingly, deletion of this sub-region or sequence thereby prevents or at least significantly reduces binding of the trans activation factor or complex to the activation region of the SUSIBA1 p.

In another embodiment, the activation region is deleted from the nucleotide sequence. In this embodiment, the genomic nucleotide sequence encoding the SUSIBA2 transcription factor lacks the activation region of the SUSIBA1 p. This total removal of the activation region thereby effectively prevents the trans activation factor or complex from binding to the SUSIBA1.

The activation region of the SUSIBA1 p in rice is shown here below (SEQ ID NO: 58):

ATTTCCTTGCTAGGTGAGACTTGAGTGGTGCTAGTCTGGCTGCAAATTT
ATAGAAGTATGTGAAAATTTGAGGTCAGAATACAAGTAATTGAATGGAC
CAATCTAATGAGTTCTGTAGCTTTAGAATAATTAATGTTAACATAAAAA
TATGTTCATGAAATCAGGTCCTTCTGCATTTTGTTGTTAACCGAATTCC
ACATTCTTCTTTAGTTCTCACAAGTACAGACAAGTATCTTGTAATGGTG
GATTCTTTTTTGGAAAACAAACTTCATTACATATTTTGTGTGATCCATC
TATGCCTTGTGCCCTTGTTACCTTTTTTTCCCTACACCTTGTTTTCTCT
TGTACTTAGTTTTGCATTGTATAACCTTTTGCTGTACTCGTGTCTTGTA
CTGTAG

The wild-type SUSIBA1 p typically comprises a sugar repressive region in addition to the activation region. In an embodiment, the genomic nucleotide sequence encoding the SUSIBA2 transcription factor also lacks at least a portion of the sugar repressive region of the SUSIBA1 p present in the intron of the wild-type version of the genomic nucleotide sequence encoding the SUSIBA2 transcription factor.

Thus, the SUSIBA1 p comprises, in an embodiment, two control elements: the activation region and the sugar repressive region. These two control elements are present in the portion of the nucleotide sequence encoding the SUSIBA2 transcription factor corresponding to an intron. These control elements are thereby part of the intronic portion of the SUSIBA1 p. The SUSIBA1 p also comprises an exonic portion present in an exon of the nucleotide sequence encoding the SUSIBA2 transcription factor.

In an embodiment, a portion of the sugar repressive region of the SUSIBA1 p is deleted from the nucleotide sequence encoding the SUSIBA2 transcription factor. In another embodiment, the sugar repressive region is deleted from the nucleotide sequence.

The deletion of the sugar repressive region or at least a portion thereof can be performed using, for instance, CRISPR/Cas9 mediated deletion or another technology, such as described in the foregoing for the activation region.

The deletion of a portion of or the complete sugar repressive region of the SUSIBA1 p is in addition to the deletion of a portion of or the complete activation region of the SUSIBA1 p.

In an embodiment, the genomic nucleotide sequencing encoding the SUSIBA2 transcription factor lacks i) at least a portion of the activation region, ii) the complete activation region, iii) at least a portion of the activation region and at least a portion of the sugar repressive region, iv) at least a portion of the activation region and the complete sugar repressive region, v) the complete activation region and at least a portion of the sugar repressive region, or vi) the complete activation region and the complete sugar repressive region of the SUSIBA1 p.

The sugar repressive region of the SUSIBA1 p in rice is shown here below (SEQ ID NO: 59):

GTATGGATCCTTTCTTTGAGTGATTACCTGGTATCGTGTAATTCTTCAT
TTGTGTATACTGTATTTGAGAGTTTGAAAAAATTTCCATAGAAAATAAT
AACATTTGTTGTTTACAAATGGTCCCGCCAAAACAGTGGAATTTATATT
GGGGATGTACATAAAAGGAGTGTAAAGTTCTAATGTGCTTATGCTAACT
TCCTTTCCATGATCTAAAGTTGTTACCTTACGGTATGCTATTTATTGGA
TCTATATTGCATTTTACTTGGTAAATCTATCTGAGGTTCCAGCTTTTGA
TATTTAAGTTTTCCTATGTTTAATTCAAAATATTCTCACGTGAATCGCA
AACCTCACCAGGAGTACAATAAATTCGTTTTATTATTATTGTAGGCTGT
GTTATTTCTAGTCCATGGTTCGGTGTCTTGAAATTTCAGTGCCAAAATT
GGGATGGATCTGGTTACATCTTCAAGTCTAATAAATGATCACACCGACT
TTATTGTGTGATTTGATTATAGCAGGGTCTTGCAACATAAATACAAGCT
ATTAATTGTGAAAGGAGAAATGAGATCTTTGGTGAGATCATGAGAATAG
GGTATAACAGACACAAT

The sugar repressive region in rice comprises a second, following portion having high sequence identity with the corresponding sugar repressive region in barley and a first, preceding portion that is not present in barley.

The activation region and the sugar repressive region of the SUSIBA1 p are both present in an intron of the SUSIBA2 gene. In an embodiment, this intron is deleted from the SUSIBA2 gene. Thus, in this embodiment, the genomic nucleotide sequence encoding the SUSIBA2 transcription factor lacks the intron comprising the activation region and the sugar repressive region of the SUSIBA1 p. In a particular embodiment, the genomic nucleotide sequence encoding the SUSIBA2 transcription factor lacks intron 2.

In an embodiment, the genomic nucleotide sequence encoding the SUSIBA2 transcription factors lacks an intronic portion of the SUSIBA1 p. In barley, intron 2 consists of the activation region and the sugar repressive region, i.e., the intronic portion of the HvSUSIBA1 p occupies intron 2. The corresponding intron 2 in rice comprises an activation region and a sugar repressive region with high sequence identity to the corresponding regions in barley. Intron 2 in rice, however, also comprises a nucleotide sequence preceding the activation region having high sequence identity to the barley activation region.

This preceding nucleotide sequence could be part of a larger activation region in rice, constitute another region within the SUSIBA1 p in rice or not forming part of the SUSIBA1 p. Hence, in an embodiment the intron may comprise nucleotide sequence(s) other than the intronic portion of the SUSIBA1 p. In such an embodiment, the intron consists of the intronic portion of the SUSIBA1 p, preferably the activation region and the sugar repressive region, and at least one other nucleotide sequence. In the present embodiment, the intronic portion of the SUSIBA1 p is deleted from the wild-type version of the genomic nucleotide sequence encoding the SUSIBA2 transcription factor. This means that following deletion of the intronic portion, the genomic nucleotide sequence encoding the SUSIBA2 transcription factor may lack intron 2, if the intronic portion occupies the complete sequence of intron 2, or may lack a portion of intron 2, if the intronic portion occupies a portion of the complete sequence of intron 2.

The nucleotide sequence of the SUSIBA1 p in rice is presented below (SEQ ID NO: 60). The underlined portion of the nucleotide sequence corresponds to the part of the SUSIBA1 p present in intron 2 of the SUSIBA2 gene. The underlined and italic portion of the nucleotide sequence corresponds to the activation region, whereas the underlined and bold portion of the nucleotide sequence corresponds to the sugar repressive region. The preceding nucleotide sequence is shown in the underlined, bold and italic portion. The remaining portion of the nucleotide sequence corresponds to the portion of the SUSIBA1 p present in exon 3 of the SUSIBA2 gene.

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

g
tgtcttgaaatttcagtgccaaaattgggatgg
atctggttacatcttcaagtctaataaatgatc
acaccgactttattgtgtgatttgattatagca
gggtcttgcaacataaatacaagctattaattg
tgaaaggagaaatgagatctttggtgagatcat
gagaatagggtataacagacacaatatttcctt
gctaggtgagacttgagtggtgctagtctggct
gcaaatttatagaagtatgtgaaaatttgaggt
cagaatacaagtaattgaatggaccaatctaat
gagttctgtagctttagaataattaatgttaac
ataaaaatatgttcatgaaatcaggtccttctg
cattttgttgttaaccgaattccacattcttct
ttagttctcacaagtacagacaagtatcttgta
atggtggattcttttttggaaaacaaacttcat
tacatattttgtgtgatccatctatgccttgtg
cccttgttacctttttttccctacaccttgttt
tctcttgtacttagttttgcattgtataacctt
ttgctgtactcgtgtcttgtactgtaggcttct
gctatcaatgatcccaaaaagcatgaaacttct
atgaaaaatgaaagcctgaatactgccctgtca
tctgacgatatgatgatcgacaatatacctcta
tgttctcgtgagtcaactctcgcagtcaatatt
tcaagtgccccgagccaactggttggaatggtt
ggtttaactgacagctcacctgctgaagttggt
acatctgagttgcatcagatgaatagctctgga
aatgctatgcaggagtcacagcctgaaagtgtg
gctgaaaagtctgcagaggatggttataactgg
cgcaaatatgggcaaaagcatgttaagggaagt
gagaacccgagaagctattacaagtgcacacat
cctaactgtgat

The genomic nucleotide sequence then preferably encodes a SUSIBA2 transcription factor (OsSUSIBA2 TF) that lacks at least a portion of the activation region of a SUSIBA1 p (OsSUSIBA1 p) present in an intron of a wild-type version of the genomic nucleotide sequence encoding the SUSIBA2 transcription factor (OsSUSIBA2 TF).

The rice plant material lacking the above mentioned activation region of the SUSIBA1 p also has low methane emission. Expression of barley SUSIBA2 (HvSUSIBA2) transcription factor in rice has been shown to lead to high starch synthesis but also low methane emissions and decrease in rhizospheric methanogen levels. Such a rice variety is, however, a transgenic rice variety comprising coding sequence of the barley SUSIBA2 transcription factor operatively connected to the barley SBEIIb promoter. The resulting transgenic rice variety thereby comprises a transgenic version of a non-genomic nucleotide sequence encoding the HvSUSIBA2 transcription factor and a genomic endogenous nucleotide sequence encoding the OsSUSIBA2 transcription factor. This genomic endogenous nucleotide sequence encoding the rice SUSIBA2 transcription factor comprises the complete sequence of the rice SUSIBA1 promoter (OsSUSIBA1 p) including its activation region and sugar repressive region.

The terms “overexpress” or “overexpression” as used herein refer to higher levels of activity of a gene, e.g., transcription of the gene; higher levels of translation of mRNA into protein; and/or higher levels of production of the gene product than would be in a rice plant material, such as in a rice cell, in its native or wild-type state. These terms can also refer to an increase in the number of copies of a gene and/or an increase in the amount of mRNA and/or gene product in the rice plant material, such as the rice cell. Overexpression can result in levels that are 25%, 50%, 100%, 200%, 500%, 1000%, 2000% or higher in the rice cell, as compared to control levels.

A “promoter” is a nucleotide sequence that controls or regulates the transcription of a nucleotide sequence, i.e., a coding sequence, which is operably associated with the promoter. The coding sequence may encode a polypeptide. Typically, a promoter refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression. Promoters can include, for example, constitutive, inducible, temporally regulated, developmentally regulated, chemically regulated, tissue-preferred and/or tissue-specific promoters.

“Operably linked” or “operably associated” as used herein means that the indicated elements are functionally related to each other, and are also generally physically related. Thus, the term operably linked or operably associated refers to nucleotide sequences on a single nucleic acid molecule that are functionally associated. Thus, a first nucleotide sequence that is operably linked to a second nucleotide sequence, means a situation where the first nucleotide sequence is placed in a functional relationship with the second nucleotide sequence. For instance, a promoter is operably associated with a nucleotide sequence if the promoter effects the transcription or expression of the nucleotide sequence, i.e., the nucleotide sequence is under transcriptional control of the promoter. Those skilled in the art will appreciate that the control sequences, e.g., promoter, need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the nucleotide sequence can still be operatively linked and under transcriptional control of a promoter.

A “heterologous” as used herein with respect to a nucleotide sequence or a gene is a nucleotide sequence or a gene not naturally associated with a rice plant material, such as a host rice cell, into which it is introduced, including non-naturally occurring multiple copies of a naturally occurring gene. A heterologous nucleotide sequence or gene may optionally be codon optimized for expression in cultivated rice according to techniques well known in the art and as further described herein. A heterologous gene also encompasses, in some embodiments, an endogenous gene controlled by a heterologous promoter and/or control elements to achieve an expression of the gene that is higher, i.e., so-called overexpression, than normal or baseline expression of the gene in rice comprising the endogenous gene under control of wild type (endogenous) promoter and control elements.

As used herein, the term “endogenous”, when used with respect to a nucleotide sequence or a gene, refers to a nucleotide sequence or gene that occurs naturally as part of the genome of a rice plant material where it is present. An endogenous nucleotide sequence or gene is sometimes referred to as a native or wild-type nucleotide sequence or gene herein.

A “genomic nucleotide sequence” refers to a nucleotide sequence present in the genome of a rice plant material, preferably in a chromosome of the rice plant material.

A “wild-type version” of a genomic nucleotide sequence refers to a non-modified genomic nucleotide sequence naturally occurring in a rice plant material. This is compared to a genomic nucleotide sequence that has been modified, such as by removal of part of the wild-type version of the genomic nucleotide sequence from the genome of the rice plant material.

A “rice plant material” is in an embodiment a rice plant. In another embodiment, a rice plant material is a rice cell, including multiple such rice cells. A rice plant material is, in a further embodiment, a rice plant tissue or organ, including but not limited to, epidermis; ground tissue; vascular tissue, such as xylem or phloem; meristematic tissues, such as apical meristem, lateral meristem or intercalary meristem; permanent tissues, such as simple permanent tissue, including for instance parenchyma, collenchyma, sclerenchyma or epidermis, complex permanent tissue, including for instance xylem, phloem, or special or secretory tissues. A rice plant material is, in yet another embodiment, a rice seed.

“Sequence identity” refers to sequence similarity between two nucleotide sequences or two peptide or protein sequences. The similarity refers to the extent to which two optimally aligned nucleotide, peptide or protein sequences are invariant throughout a window of alignment of nucleotides or amino acids. Identity can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An identity fraction for aligned segments of a test sequence and a reference sequence is the number of identical nucleotides or amino acids which are shared by the two aligned sequences divided by the total number of nucleotides or amino acids in the reference sequence segment, i.e., the entire reference sequence or a smaller defined part of the reference sequence. Percent sequence identity is represented as the identity fraction multiplied by 100.

An embodiment relates to a method of improving resistance of a rice plant material against a biotic stress. The method comprises overexpressing a FatB gene in the rice plant material.

In an embodiment, overexpressing the FatB gene comprises replacing a promoter of the FatB gene, or at least a portion thereof, by a promoter selected from the group consisting of an ARP1 promoter, an H3F3 promoter, an HSP promoter, an H2BF3 promoter, a CaMV 35S promoter, a barley SBEIIb promoter and a heterologous FatB promoter.

In an embodiment, the rice plant material is an O. sativa plant material or an O. glaberrima plant material. In a particular embodiment, overexpressing the FatB gene comprises replacing a promoter of an O. sativa or O. glaberrima FatB gene by an O. eichigeri FatB promoter.

In an embodiment, the biotic stress is rice brown planthopper and/or rice blast fungus.

### EXAMPLE

This example shows that a single gene of rice FatB6 confers resistance to rice brown planthopper and rice blast fungus. Wild rice (Oryza eichigeri) has high oil (triacylglycerol) content in the leaves, leaf sheath and stems compared with Nipponbare (Oryza sativa, Nipponbare). The oil content in wild rice was associated with high expression of the FatB6 gene. Overexpression of the FatB6 gene in Nipponebare by stable transformation led to high oil content in Nipponbare leaves, leaf sheath and stems. Importantly, the transformed rice with high oil content showed significant resistance against rice brown planthopper and rice blast fungus. Hence, the FatB6 gene plays an important role in wild rice resistance against rice brown planthopper and rice blast fungus via high oil content. The gene can be employed in breeding to raise resistance against biotic stress factors of insect pests and diseases.

Materials and Methods

Plant Materials and Growth Conditions

Rice plants of wild rice (Oryza eichigen), variety Nipponbare (Oryza sativa L. ssp. Japonica) and transformed lines were grown in a phytotron, greenhouse or open fields. Open field cultivation was performed in a similar way to that described previously (Zhang et al. 2012). Phytotron conditions were applied to mimic field conditions, but with limited high temperatures. In the phytotron, rice plants were grown in cylinder-type pots (30 cm high with an upper diameter of 29 cm and bottom diameter of 19 cm) with organic soil containing plant residues. Phytotron growth management was similar to that described previously (Nalawade et al. 2012) with a modified setting for rice, i.e., 14 h light/10 h dark at 30° C./21° C., a constant relative humidity of 80% and light intensity of 400 μmol photons m⁻² s⁻¹.

Oligonucleotides

The oligonucleotides used in this example are listed in Table 1 and were purchased from Sigma-Aldrich (St. Louis, Mo., USA).

TABLE 1
oligonucleotides

| | | | |
|---|---|---|---|
| | | | SEQ |
| | Gene or | Se- | ID |
| Oligo name | promoter | quence | NO: |

Primers used for qPCR

| | | | |
|---|---|---|---|
| qOsWRI1F | OsWRI1 | GCGGT | 1 |
| | | AACCA | |
| | | ACTTC | |
| | | GACAT | |
| qOsWRI1R | | CTGCA | 2 |
| | | TTCTC | |
| | | ACTTC | |
| | | GGTCA | |
| qOsOLEF | OsOleosion | CCGCG | 3 |
| | | CTCTC | |
| | | CGTGT | |
| | | TCTC | |
| qOsOLER | | GTGCT | 4 |
| | | GCGCC | |
| | | GCCTC | |
| | | CTT | |
| qOsCaIF | OsCaleosion | TCGGA | 5 |
| | | TGGTT | |
| | | CGCGG | |
| | | CGAAG | |
| qOsCaIR | | GTCGT | 6 |
| | | ACATG | |
| | | CGCCG | |
| | | GATGG | |
| qPKcyto-1F | OsPK- | TTCTG | 7 |
| | cytoplasm | CCAAA | |
| | | GCCAC | |
| | | CGATT | |
| | | C | |
| qPKcyto-1R | | ACGGA | 8 |
| | | TGCGA | |
| | | CGCCA | |
| | | ATACG | |
| qNFatB6F | NippFatB6 | CCTCC | 9 |
| | | ATCCA | |
| | | GTGTG | |
| | | ACCAT | |
| | | C | |
| qNFatB6R | | AGCCC | 10 |
| | | ATGTT | |
| | | CCCCT | |
| | | CGCCC | |
| qNFatB2F | NippFatB2 | CGGTG | 11 |
| | | CCTCA | |
| | | CAGTG | |
| | | CTCCA | |
| qNFatB2R | | AACAC | 12 |
| | | CATAC | |
| | | CGTCC | |
| | | TGGAT | |
| | | G | |
| qNFatB11F | NippFatB11 | CACCA | 13 |
| | | GCATT | |
| | | GGCGC | |
| | | CGACA | |
| qNFatB11R | | GCGTT | 14 |
| | | CTCAG | |
| | | CTGCT | |
| | | GCGTG | |
| qJSFatB4F | OeFatB4 | GAGCT | 15 |
| | | GAAAT | |
| | | AGGCC | |
| | | CGTAC | |
| qJSFatB4R | | GAGGA | 16 |
| | | TTCTT | |
| | | TGTTG | |
| | | CCATC | |
| | | G | |
| qJSFatB6F | OeFatB6 | ATAGG | 17 |
| | | CCCGT | |
| | | ACTTC | |
| | | AATGG | |
| | | TT | |
| qJSFatB6R | | GAGAA | 18 |
| | | CCAGC | |
| | | CATCC | |
| | | ATCCG | |
| qJSFatB8F | OeFatB8 | GCTGC | 19 |
| | | TACCA | |
| | | AACAA | |
| | | TTCAC | |
| | | AA | |
| qJSFatB8R | | ACTCC | 20 |
| | | AGCTG | |
| | | AAGCA | |
| | | GATGG | |
| | | TT | |

Primers used for cloning and
vector construction

| | | | |
|---|---|---|---|
| NFatBchr2FXbaI | NippFatB2 | GCTCT | 21 |
| | | AGAAT | |
| | | GGCAG | |
| | | GGTCT | |
| | | CTTGC | |
| | | CGCC | |
| NFatBchr2REcoR1 | | CGGAA | 22 |
| | | TTCCT | |
| | | AGGCT | |
| | | AACTT | |
| | | TTCAC | |
| | | TCTG | |
| NFatBchr6FXbaI | NippFatB6 | GCTCT | 23 |
| | | AGAAT | |
| | | GGCTG | |
| | | GTTCT | |
| | | CTTGC | |
| | | GGC | |
| NFatBchr6REcoR1 | | CGGAA | 24 |
| | | TTCTC | |
| | | ATGCA | |
| | | CTCTC | |
| | | AGCTG | |
| | | GGA | |
| NfatBchr11FXbaI | NippFatB11 | GCTCT | 25 |
| | | AGAAT | |
| | | GGCAG | |
| | | GGTCT | |
| | | CTTGC | |
| | | CGCC | |
| NFatBchr11REcoR1 | | CGGAA | 26 |
| | | TTCTT | |
| | | ACGCG | |
| | | TTCTC | |
| | | AGCTG | |
| | | CTGCG | |
| SBEIIbFXbaI | Barley | GCTCT | 27 |
| | SBEIIb | AGACT | |
| | promoter | GCAGG | |
| | | TCAAC | |
| | | GGATC | |
| | | CTT | |
| SBEIIbRXbaI | | GCTCT | 28 |
| | | AGAAG | |
| | | TTCTA | |
| | | TTTCA | |
| | | CTCAG | |
| | | GGT | |
| jsFatBFXbaIcom | OeFatB | TTTCT | 29 |
| | | AGAAT | |
| | | GGCTG | |
| | | GTTCT | |
| | | CTTGC | |
| | | GGC | |
| jsFatBREcoR1com | | ATGAA | 30 |
| | | TTCTT | |
| | | GCCGG | |
| | | ATAAA | |
| | | CTACA | |
| | | GAA | |
| pNFatB2F | NippFatB2 | GTACA | 31 |
| | promoter | TGTAG | |
| | | GTCTT | |
| | | GTTTA | |
| pNFatB2R | | CTTCT | 32 |
| | | AGCTG | |
| | | ATGCT | |
| | | GCAGG | |
| pNFatB6F | NippFatB6 | ACAGA | 33 |
| | promoter | AATTT | |
| | | CGCTG | |
| | | GCCAT | |
| pNFatB6R | | CTGGC | 34 |
| | | AATTC | |
| | | ACCGG | |
| | | TTGTG | |
| pNFatB11F | NippFatB11 | TTCTC | 35 |
| | promoter | GTATC | |
| | | CTAGC | |
| | | CCATA | |
| pNFatB11R | | CTTCT | 36 |
| | | AGCTG | |
| | | ATGCT | |
| | | GCAGG | |
| pjsFatB4F | OeFatB4 | ACAGA | 37 |
| | promoter | AATTT | |
| | | CGCTG | |
| | | GCCAT | |
| | | G | |
| pjsFatB4R | | CCACA | 38 |
| | | GACAC | |
| | | TCAAA | |
| | | TTCTC | |
| pjsFatB6F | OeFatB6 | ACAGA | 39 |
| | promoter | AATTT | |
| | | CGCTG | |
| | | GCCAT | |
| | | G | |
| pjsFatB6R | | CCACA | 40 |
| | | GACAC | |
| | | TCAAA | |
| | | TTCTC | |

Gene Expression Analysis by Quantitative Polymerase Chain Reaction (qPCR)

RNA isolation, cDNA synthesis and qPCR analysis were performed in accordance with previous reports (Sun et al. 2005; Zhang et al. 2012; Jin et al. 2017a). In brief, plant materials from different tissues were ground into fine powders in liquid nitrogen and total RNA was isolated by the Spectrum™ Plant Total RNA Kit (Sigma-Aldrich, St. Louis, Mo., US) according to the manufacturer protocol using 30 mg plant materials. All samples were treated with DNase I (Sigma-Aldrich, St. Louis, Mo., US) to remove trace amounts of DNA contamination. Total RNA of 1 μg was used as a template for the cDNA synthesis with the Quanta qScript cDNA synthesis kit (Quanta Biosciences, Gaitherburg, Md., USA). The synthesized cDNA was adjusted to a concentration of 5 ng/μl and 15 ng was used for qPCR analysis. qPCR reactions with at least 90% amplification efficiency were performed in a volume of 20 μl containing 5 μM specific primers and a SYBR Green PCR master mix (Applied Biosystems, Life Technologies Europe BV, Stockholm, Sweden). The PCR program consisted of an initial temperature of 95° C. for 4 min, and then 35-40 cycles of 30 seconds at 95° C. and 30 seconds at 60° C. The melt curve was performed by increasing the temperature from 60° C. to 95° C. at a speed of 0.05° C. per second. qPCR-specific amplification was verified by a single band product in gel analysis. Data was calculated with the comparative Ct method (Zhang et al. 2012) and one-way ANOVA (Zhang et al. 2012) was used for statistical analysis. The gene expression level by qPCR was normalized using Ubiquitin10 (Jain et al. 2006).

Rice Genomic DNA Isolation and Promoter Sequence Analysis

Rice genomic DNA was isolated from leaves using a CTAB method as described (Su et al. 2015). The promoter regions of Nipponbare. Jinsui (Oryza eichingen), Duanhua (Oryza brachyantha), and CCDD (Oryza latifolia) were amplified by PCR (see Table 1 for primers) and analyzed by DNASTAR lasergene 14.

Plasmid Construction and Rice Transformation

Plasmid construction and general molecular cloning procedures were performed according to previously developed protocols (Sun et al. 2003; Sun et al. 2005; Sun et al. 1998). Different FatB genes from wild rice and Nipponbare were cloned and fused to nucleotides 1-936 of barley SBEIIb promoter (HvSBEIIb p; Genbank Accession No AF064563). The fused DNA fragment was cloned in the pCAMBIA 1301 binary vector. The plasmid construct was used for Agrobacterium-mediated transformation of rice following a published protocol (Hiei et al. 1994). Screening of post-transformants was based on hygromycin resistance and PCR determination of T-DNA insertion. A To line of fused barley SBEIIb promoter and Nipponbare FatB6 line was used for detailed studies of oil content and resistance against rice brown planthopper and rice blast fungi. A binary vector containing HvSBEIIb p:GUS was also constructed and transformed to Nipponbare. All final constructs were verified by DNA sequencing at Macrogen Europe (Amsterdam, the Netherlands), and transformed into Agrobacterium tumefaciens strain EHA105 before agro-transformation into rice.

Observation of Oil Abundance and Determination of Oil Content in Rice

For observation of oil abundance, rice leaves or leaf sheath were detached at 3 μm and incubated for 15 min in a 1×PBS phosphate-buffered saline (PBS) solution pH 7.4 containing 4% formaldehyde under a vacuum condition to fix oil bodies in the tissue cells. Then the tissue was stained for 20 min with a dye solution of 25 μg ml⁻¹ Nile Red in 1×PBS under vacuum. After wash with 1×PBS three times, the tissue was placed on a slide for fluorescent observation of oil droplets under a confocal microscope with an excitation light of 488 nm. For determination of oil content, a protocol of oil extraction, thin layer chromatography (TLC) separation and gas chromatography (GC) measurements was followed and performed according to Aslan et al. (2015) and Jin et al. (2017b).

Examination of Resistance Against Rice Brown Planthopper (Nilaparvata lugens)

The rice brown planthopper used for inoculation were collected from rice fields in Zhejiang Province, China, and maintained on TN1 plants in a phytotron with a condition of 12 h light (270 μmol photons m⁻² s⁻¹)/12 h darkness at 26° C. and a relative humidity of 70%. The resistance to rice brown planthopper of transgenic rice plants was essentially evaluated by host choice test as previously described by Du et al. (2009) with appropriate modifications. One 4 month-old transgenic rice plant was placed with one control plant of the same stage in a net chamber with 12 h light (270 μmol photons m⁻² s⁻¹)/12 h darkness at 26° C. The rice plants were infested with rice brown planthopper at the rate of approximately 2 instar nymphs and 2 adults per tiller. Numbers of rice brown planthopper on each tiller of transgenic rice or Nipponbare were recorded at 2, 7, 14, 21, 28, 35 and 44 days post infestation. Biological triplicate experiments were carried out.

Examination of Resistance Against Rice Blast Fungus (Magnaporthe oryzae)

The M. oryzae pathogens were originally collected and isolated from rice fields in Zhejiang Province and cultured in potato dextrose agar (PAD) medium at 25° C. before used for inoculation. Rice blast fungus inoculation was carried out as described previously (Li et al. 2010) with minor modifications. Leaf fragments were cut from six to eight week-old rice plants of transgenic lines and controls and placed in plastic plates covered by wet filters at the leaf fragment ends. Droplets (10 μl) of M. oryzae spore suspension (approximately 1×10⁵ spores/ml) were inoculated carefully on the leaf surfaces. Inoculated leaves were kept in a growth chamber with 12 h light/12 h darkness at 26° C. Lesion symptoms and sizes were photographed and measured at 3-8 days post inoculation.

Results and Discussion

More Oil (Triacylglycerol) in Leaves and Stems of Wild Rice (Oryza eichigeri) than Nipponbare

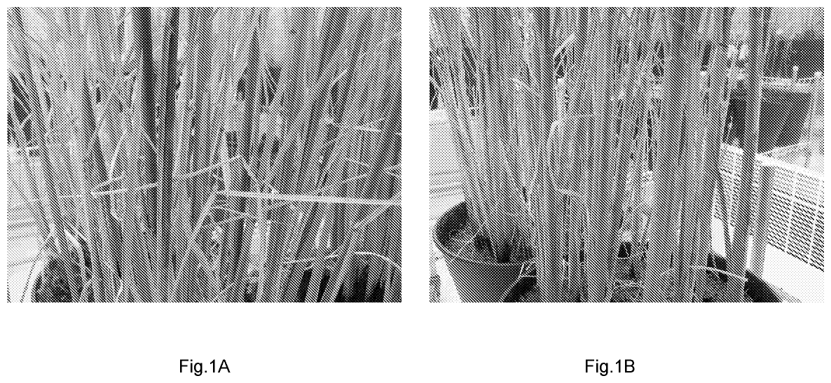

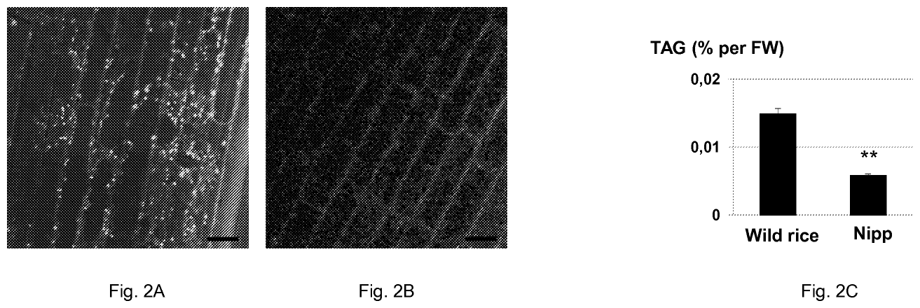

The phenotypic trait of wild rice leaves and stems are similar to Nipponbare except that the wild rice may have more pigments in their leaf sheath, see FIGS. 1A and 1B. The oil content in leaf sheath and stems were examined by a confocal microscope after the Nile Red staining and by GC quantification after TLC separation. The confocal microscope image showed that wild rice cells of leaf sheath have more oil droplets than Nipponare, see FIGS. 2A and 2B, and the GC quantitation, see FIG. 2C, demonstrated that oil content in wild rice leaf sheath and stems was significantly higher than in Nippon bare.

The High Oil Content in Wild Rice was Associated with High Expression of FatB6 in Wild Rice

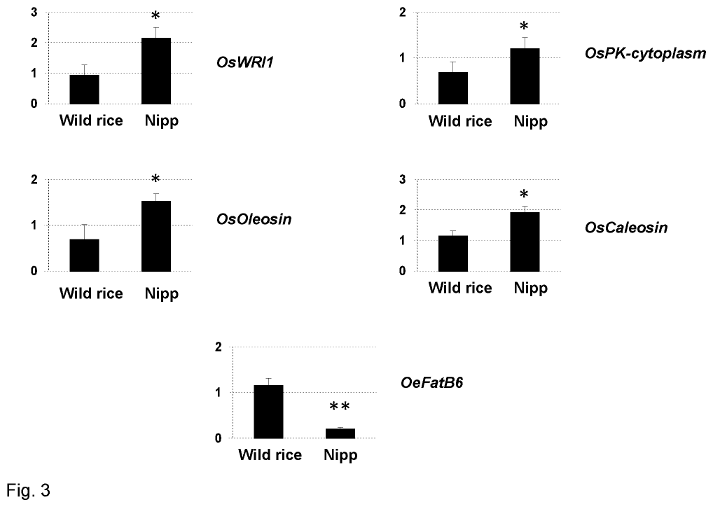

To unravel which gene was responsible for the high oil content in wild rice, five key genes that are involved in oil formation in wild rice were screened by qPCR, see FIG. 3. Interestingly, high expression of FatB6 was associated with the high oil content in the tissue of wild rice. Since the genome sequence of Japonica rice is available, all three FatB cDNAs were cloned in Nipponbare using that genome sequence. These genes were located in Japonica rice chromosome 2, 6 and 11, respectively, and therefore defined as Nipponbare rice FatB2 (NippFatB2), FatB6 (NippFatB6) and FatB11 (NippFatB11).

NippFatB2 cDNA
(SEQ ID NO: 41)
atggcagggtctcttgccgcctcagcattct
tcccaggtccaggctcatctcctgcagcatc
agctagaagctccaagaatgctgctgttacc
ggcgaattgccggagaatttgagtgtctgtg
gcattgtcgcaaagcctaacccacctcctgc
agccatgcaagtaaaggcacaggctcaaacc
cttcccaaggttaatggtacgaaggttaacc
tcaagacggtgaagcctgacatggaggaaac
ggtgcctcacagtgctccaaagacgttctat
aaccaactgccggattggagcatgcttcttg
cggctattacaaccatcttcctcgccgcaga
gaagcagtggacactgcttgattggaagccg
aagaaacctgacatgcttgttgacacatttg
gctttggtaggatcatccaggacggtatggt
gtttaggcagaacttcatgattcggtcctac
gagattggcgctgatcgtacagcttctatag
agacattgatgaatcatttacaggaaacggc
tcttaaccatgtaaggactgctggtcttctt
ggagatggttttggggctacaccggagatga
gcaaacggaacttgatatgggttgtcagcaa
aatccagcttcttgttgagcaataccccgca
tggggagatatggttcaagttgacacatggg
tcgctgctgctggcaaaaatggcatgcgtcg
agactggcatgttcgtgactacaactctggc
cgaacaatcttgagagctacaagtgtttggg
tgatgatgcacaagaaaactagaagactttc
aaaaatgccagatgaagttagagctgaaata
ggcccatatttcaatgaccgttcagctataa
cagaggagcagagtgaaaagttagcctag
NippFatB2 peptide
(SEQ ID NO: 42)
MAGSLAASAFFPGPGSSPAASARSSKNAAVT
GELPENLSVCGIVAKPNPPPAAMQVKAQAQT
LPKVNGTKVNLKTVKPDMEETVPHSAPKTFY
NQLPDWSMLLAAITTIFLAAEKQWTLLDWKP
KKPDMLVDTFGFGRIIQDGMVFRQNFMIRSY
EIGADRTASIETLMNHLQETALNHVRTAGLL
GDGFGATPEMSKRNLIWWSKIQLLVEQYPAW
GDMVQVDTWVAAAGKNGMRRDWHVRDYNSG
RTILRATSVWVMMHKKTRRLSKMPDEVRAEI
GPYFNDRSAITEEQSEKLA
NippFatB6 cDNA
(SEQ ID NO: 43)
atggctggttctcttgcggcgtctgcattct
tccctgtcccagggtcttcccctgcagcttc
ggctagaagctctaagaacacaaccggtgaa
ttgccagagaatttgagtgtccgcggaatcg
tcgcgaagcctaatccgtctccaggggccat
gcaagtcaaggcgcaggcgcaagcccttcct
aaggttaatggaaccaaggttaacctgaaga
ctacaagcccagacaaggaggatataatacc
gtacactgctccgaagacattctataaccaa
ttgccagactggagcatgcttcttgcagctg
tcacgaccattttcctggcagctgagaagca
gtggactctgcttgactggaagccgaagaag
cctgacatgctggctgacacattcggctttg
gtaggatcatccaagacgggctggtgtttag
gcaaaacttcttgattcggtcctacgagatt
ggtgctgatcgtacagcttctattgagacat
taatgaatcatttacaggaaacagctctgaa
ccatgtgaaaactgctggtctcttaggtgat
ggttttggtgctacgccggagatgagcaaac
ggaacttaatatgggttgtcagcaaaattca
gcttcttgttgagcgatacccatcatgggga
gatatggtccaagttgacacatgggtagctg
ctgctggcaaaaatggcatgcgtcgagattg
gcatgttcggaactacaactctggtcaaaca
atcttgagggctacaagtgtttgggtgatga
tgaataagaacactagaagactttcaaaaat
gccagatgaagttagagctgaaataggcccg
tatttcaatggccgttctgctatatcagagg
agcagggtgaaaagttgcctaagccagggac
cacatttgatggcgctgctaccaaacaattc
acaagaaaagggcttactccgaagtggagtg
accttgatgtcaaccagcatgtgaacaatgt
gaagtatattggttggatacttgagagtgct
ccaatttcgatactggagaagcacgagcttg
caagcatgaccttggattacaggaaggagtg
tggccgtgacagtgtgcttcagtcgcttacc
gctgtttcaggtgaatgcgatgatggcaaca
cagaatcctccatccagtgtgaccatctgct
tcagctggagtccggagcagacattgtgaag
gctcacacagagtggcgaccgaagcgagctc
agggcgaggggaacatgggctttttcccagc
tgagagtgcatga
NippFatB6 peptide
(SEQ ID NO: 44)
MAGSLAASAFFPVPGSSPAASARSSKNTTGE
LPENLSVRGIVAKPNPSPGAMQVKAQAQALP
KVNGTKVNLKTTSPDKEDIIPYTAPKTFYNQ
LPDWSMLLAAVTTIFLAAEKQWTLLDWKPKK
PDMLADTFGFGRIIQDGLVFRQNFLIRSYEI
GADRTASIETLMNHLQETALNHVKTAGLLGD
GFGATPEMSKRNLIWVVSKIQLLVERYPSWG
DMVQVDTWVAAAGKNGMRRDWHVRNYNSGQT
ILRATSVWVMMNKNTRRLSKMPDEVRAEIGP
YFNGRSAISEEQGEKLPKPGTTFDGAATKQF
TRKGLTPKWSDLDVNQHVNNVKYIGWILESA
PISILEKHELASMTLDYRKECGRDSVLQSLT
AVSGECDDGNTESSIQCDHLLQLESGADIVK
AHTEWRPKRAQGEGNMGFFPAESA
NippFatB11 cDNA
(SEQ ID NO: 45)
atggcagggtctcttgccgcctcagcattct
tcccaggtccaggctcatctcctgcagcatc
agctagaagctccaagaatgctgctgttacc
ggcgaattgccggagaatttgagtgtccgtg
gcattgtcgcaaagcctaacccacctcctgc
agccatgcaagtaaaggcacaggctcaaacc
cttcccaaggttaatggtacgaaggttaacc
tcaagacggtgaagcctgacatggaggaaac
ggtgccttacagtgctccaaagacgttctat
aaccaactgccggattggagcatgcttcttg
cggctattacaaccatcttccttgccgcaga
gaagcagtggacactgcttgattggaagcca
aagaaacctgacatgcttgttgacacatttg
gctttggtaggattatccaggacggtatggt
gtttaggcagaacttcatgattcggtcctac
gagattggtgctgatcgtacagcttctatag
agacattgatgaatcatttacaggaaacagc
tcttaaccatgtgaggactgctggtcttctt
ggagatggttttggggctacaccggagatga
gcaaacggaacttgatatgggttgtcagcaa
aatccagcttcttgttgagcaataccccgca
tggggagatacggttcaagttgacacatggg
ttgctgctgctggcaaaaatggcatgcgtcg
agactggcatgttcgtgactacaactctggc
cgaacaatcttgagagctacaagtgtttggg
tgatgatgcacaagaaaactagaagactttc
aaaaatgccagatgaagttagagctgaaata
ggcccatatttcaatgaccgttcagctataa
cagaggagcagagtgaaaagttagccaagac
aggaaataaagttggtgatgatgctacagag
caattcataagaaaggggctcactcctagat
ggggtgacctcgatgtcaatcagcatgtgaa
caatgttaaatatattgggtggatccttgag
agtgctccaatttcagtactggagaagcatg
agcttgcaagcatgaccctggattacaggaa
ggagtgtggtcgagacagcgtgctgcaatca
cttaccaccgtgtcaggggaatgcaccagca
ttggcgccgacaagcaggcttctgccatcca
gtgcgaccatcttcttcagcttgagtcagga
gctgatattgtgaaggcacacacagagtggc
gaccaaagcgatcgcacgcagcagctgagaa
cgcgtaa
NippFatB11 peptide
(SEQ ID NO: 46)
MAGSLAASAFFPGPGSSPAASARSSKNAAVT
GELPENLSVRGIVAKPNPPPAAMQVKAQAQT
LPKVNGTKVNLKTVKPDMEETVPYSAPKTFY
NQLPDWSMLLAAITTIFLAAEKQWTLLDWKP
KKPDMLVDTFGFGRIIQDGMVFRQNFMIRSY
EIGADRTASIETLMNHLQETALNHVRTAGLL
GDGFGATPEMSKRNLIWWSKIQLLVEQYPAW
GDTVQVDTWVAAAGKNGMRRDWHVRDYNSG
RTILRATSWVMMHKKTRRLSKMPDEVRAEI
GPYFNDRSAITEEQSEKLAKTGNKVGDDATE
QFIRKGLTPRWGDLDVNQHVNNVKYIGWILE
SAPISVLEKHELASMTLDYRKECGRDSVLQS
LTTVSGECTSIGADKQASAIQCDHLLQLESG
ADIVKAHTEWRPKRSHAAAENA
Using the Nipponbare sequences, the
corresponding wild rice FatB cDNAs,
i.e., Oryza eichigeri FatB2
(OeFatB2), FatB6 (OeFatB6) and
FatB11 (OeFatBH), were also cloned.
OeFatB2 cDNA
(SEQ ID NO: 47)
atggctggttctcttgcggcgtctgcattct
tccctagcccagggtcttcccctgcagcatc
gactagaagttctaagaatacaaccagtgaa
ttgccagagaatttgagtgtccgtggaatcg
tcgcgaagcctaacccgcctccgggggccat
gcaagtcaaggcgcaagcgcaagcccttccc
aaggttaatggaaccaaggttaacctgaaga
ctacaagcccagagaaggaggatacaatacc
gtacactgctccgaagacgttctataaccaa
ctgccagactggagcatgcttcttgcagctg
tcacaaccattttcctggcagctgagaagca
atggactctgcttgactggaagccgaagaag
cctgacatgctggctgacacattcagctttg
gtaggattatccaagacgggctggtgtttag
gcaaaacttcttgattcggtcctacgagatt
ggtgctgatcgtacagcttctatagagacat
taatgaatcatttacaggaaacagctctgaa
ccatgtgaaaactgctggtctcctaggtgat
ggttttggtgctacgccggagatgagcaaac
ggaacttaatatgggttgtcagcaaaattca
gcttcttgttgagcgatacccatcatgggga
gatatggtccaagttgacacatgggtagctg
ctgctggcaaaaatggcatgcgtcgagattg
gcatgtttgtgactacaactctggtcaaaca
atcttgagggctacaagtgtttgggtgatga
tgaataagaacactagaagactttcaaaaat
gccagatgaagttagagctgaaataggcccg
tacttcaatggttgttccgctataacagagg
agcagtgtgaaaagttgcctaagccagggac
cacatttgatggcactgctaccaaacaattc
acaagaaaagggcttactccgaagtggagtg
accttgatgtcaaccagcatgtgaacaatgt
gaagtatatcggatggatggctggttctctt
gcggcgtctgcattcttccctagcccagggc
gaggggaacatgggttttttcccagctga
OeFatB2 peptide
(SEQ ID NO: 48)
MAGSLAASAFFPSPGSSPAASTRSSKNTTSE
LPENLSVRGIVAKPNPPPGAMQVKAQAQALP
KVNGTKVNLKTTSPEKEDTIPYTAPKTFYNQ
LPDWSMLLAAVTTIFLAAEKQWTLLDWKPKK
PDMLADTFSFGRIIQDGLVFRQNFLIRSYEI
GADRTASIETLMNHLQETALNHVKTAGLLGD
GFGATPEMSKRNLIWVVSKIQLLVERYPSWG
DMVQVDTWVAAAGKNGMRRDWHVCDYNSGQT
ILRATSVWVMMNKNTRRLSKMPDEVRAEIGP
YFNGCSAITEEQCEKLPKPGTTFDGTATKQF
TRKGLTPKWSDLDVNQHVNNVKYIGWMAGSL
AASAFFPSPGRGEHGFFPS
OeFatB6 cDNA
(SEQ ID NO: 49)
atggctggttctcttgcagcgtctgcattct
tccctggcccagggtcttcccctgcagcatc
agctagaagttctaagaacacaaccggtgaa
ttgccagagaatttgagtgtccgcggaatcg
ttgcgaagcctaatccgcctccgggagccat
gcaagtcaaggcgcaggcgcaagcccttcct
aaggttaatggaaccaaggttaacctgaaga
ctactagcccagacaaggaggatacaatacc
atacactgctccgaagacattctataaccaa
ttgccagactggagcatgcttcttgcagctg
tcacgaccattttcctggcagctgagaagca
atggactctgcttgactggaagccgaagaag
cctgacatgctggctgacacatttggctttg
gtaggatcatccaagatgggctggtgtttag
gcaaaacttcctgattcggtcctacgaaatt
ggtgctgatcgtacagcttctatagagacat
taatgaatcatttacaggaaacagcactgaa
ccatgtgaaaactgctggtctcctaggtgat
ggttttggtgctacgccggagatgagcaaac
ggaacttaatatgggttgtcagcaaaattca
gcttcttgttgagcgatacccatcatgggga
gatatggtccaagttgacacatgggtagctg
ctgctggcaaaaatggcatgcgtcgagattg
gcatgtttgtgactacaactctggtcaaaca
atcttgagggctacaagtgtttgggtgatga
tgaataagaacactagaagactttcaaaaat
gccagatgaagttagagctgaaataggcccg
tacttcaatggttgttccgctataacagagg
agcagtgtgaaaagttgcctaagccagggac
cacatttgatggcactgctaccaaacaattc
acaagaaaagggcttactccgaagtggagtg
accttgatgtcaaccagcatgtgaacaatgt
gaagtatattggatggatacttgagagtgct
ccaatttccatactggagaagcacgagcttg
caagcatgaccttggattacaggaaggagtg
tggccgtgacagtgtgcttcagtcacttacc
accgtatcaggtgaatgtgtcgatggcaaca
aagaatcctccatccagtgtaaccatctgct
tcagctggagtccggagcagacattgtgaag
gctcacacagagtggcgaccaaagcgagcgc
agggcgaggggaacatgggttttttcccagc
tgagagcgcatga
OeFatB6 peptide
(SEQ ID NO: 50)
MAGSLAASAFFPGPGSSPAASARSSKNTTGE
LPENLSVRGIVAKPNPPPGAMQVKAQAQALP
KVNGTKVNLKTTSPDKEDTIPYTAPKTFYNQ
LPDWSMLLAAVTTIFLAAEKQWTLLDWKPKK
PDMLADTFGFGRIIQDGLVFRQNFLIRSYEI
GADRTASIETLMNHLQETALNHVKTAGLLGD
GFGATPEMSKRNLIWVVSKIQLLVERYPSWG
DMVQVDTWVAAAGKNGMRRDWHVCDYNSGQT
ILRATSVWVMMNKNTRRLSKMPDEVRAEIGP
YFNGCSAITEEQCEKLPKPGTTFDGTATKQF
TRKGLTPKWSDLDVNQHVNNVKYIGWILESA
PISILEKHELASMTLDYRKECGRDSVLQSLT
TVSGECVDGNKESSIQCNHLLQLESGADIVK
AHTEWRPKRAQGEGNMGFFPAESA
OeFatB11 cDNA
(SEQ ID NO: 51)
atggctggttctcttgcggcgtctgcattct
tccctggcccagggtcttcccctgcagcatc
agctagaagttctaagaacacaaccggtgaa
ttgccagagaatttgagtgtccgcggaatcg
ttgcgaagcctaatccgcctccgggagccat
gcaagtcaaggcgcaggcgcaagcccttcct
aaggttaatggaaccaaggttaacctgaaga
ctactagcccagacaaggaggatacaatacc
gtacactgctccgaagacgttctataaccaa
ttgccagactggagcatgcttcttgcagctg
tcacgaccattttcctggcagctgagaagca
atggactctgcttgactggaagccgaagaag
cctgacatgctggctgacacatttggctttg
gtaggatcatccaagatgggctggtgtttag
gcaaaacttcctgattcggtcctacgaaatt
ggtgctgatcgtacagcttctatagagacat
taatgaatcatttacaggaaacagcactgaa
ccatgtgaaaactgctggtctcctaggtgat
ggttttggtgctacaccggagatgagcaaac
ggaacttaatatgggttgtcagcaaaattca
gcttcttgttgagcgatacccatcatgggga
gatatggtccaagttgacacgtgggtagctg
ctgctggcaaaaatggcatgcgtcgagattg
gcatgtacgggactacaactctggtcaaaca
atcttgagggctacaagtgtttgggtgatga
tgaataagaacactagaagactttcaaaaat
gccagatgaagttagagctgaaataggcccg
tacttcaatggtcgttctgttatcacagagg
agcagggtgaaaagttgcctaagccagggac
cacatttgatggcgctgctaccaaacaattc
acaagaaaagggcttactccaaagtggagtg
accttgatgtcaaccagcatgtgaacaatgt
gaagtatattggatggatacttgagagtgct
ccaatttcgatactggagaagcacgagcttg
caagcatgaccttggattacaggaaggagtg
tggccgtgacagtgtgcttcagtcacttacc
accgtatcaggtgaatgtgtcgatggcaaca
aagaatcctccatccagtgtaaccatctgct
tcagctggagtccggagcagacattgtgaag
gctcacacagagtggcgaccaaagcgagcgc
agggcgaggggaacatgggttttttcccagc
tgagagcgcatga
OeFatBH peptide
(SEQ ID NO: 52)
MAGSLAASAFFPGPGSSPAASARSSKNTTGE
LPENLSVRGIVAKPNPPPGAMQVKAQAQALP
KVNGTKVNLKTTSPDKEDTIPYTAPKTFYNQ
LPDWSMLLAAVTTIFLAAEKQWTLLDWKPKK
PDMLADTFGFGRIIQDGLVFRQNFLIRSYEI
GADRTASIETLMNHLQETALNHVKTAGLLGD
GFGATPEMSKRNLIWVVSKIQLLVERYPSWG
DMVQVDTWVAAAGKNGMRRDWHVRDYNSGQT
ILRATSVWVMMNKNTRRLSKMPDEVRAEIGP
YFNGRSVITEEQGEKLPKPGTTFDGAATKQF
TRKGLTPKWSDLDVNQHVNNVKYIGWILESA
PISILEKHELASMTLDYRKECGRDSVLQSLT
TVSGECVDGNKESSIQCNHLLQLESGADIVK
AHTEWRPKRAQGEGNMGFFPAESA

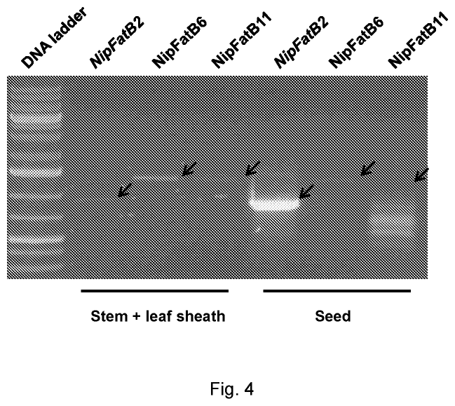

When expression of all three NippFatB was analyzed in the stems and leaf sheath of Nipponbare, expression of all three FatB genes were very low in the Nipponbare tissues except a slightly high expression of NippFatB6, see FIG. 4. The low oil content in Nipponbare rice might be due to low expression of the FatB genes. Thus, the promoter regions of the FatB genes in Nipponbare and wild rice were cloned to examine if the promoter sequences were different, which led to different expressions of FatB in wild rice and in Nipponbare, particularly for FatB6. Using PCR cloning, the promoter sequences of NippFatB2, NippFatB6 and NippFatB11 and OeFatB2 and OeFatB6 were successfully obtained. Underlined nucleotides in SEQ ID NO: 53-57 indicate open reading frame.

NippFatB2 promoter
(SEQ ID NO: 53)
gtacatgtaggtcttgtttagatcccaaaaa
attttagccaaaacctcacatcaaatatttg
gacacatgcacccctaccagtgtgtggaggc
attgcatacacgaaacatggaaaaggaatca
acttgagaggttagacctgctagctctacta
ggtctggatggtcatgcatttttttttgaaa
aaaaccacgctgcaagctcgacagcctcaac
ctcaatggcaaccatgacaataatatgcatg
acaatggtgtaggagaaaagacacgtcgata
accaaagggcgcggctgcgcatacaaaggcg
gagagaaggaacgatggtggctcaaaaagaa
agagcgtcggtggcagtggtgcgtggagcga
cactaaagttagtggttgctgatggtctcac
acaatccctaatcgaaatatttatttttttt
cacttagtattgctgatccgtgggccaccag
ccaatcataaagaaaaatgttgagataaaag
gtggagtatcttccccttccttccctttttg
actcgaaaaaaaaaagcgtcggtggcggccg
tgcgtgtaacaacactaaagttagtggttgc
tggtggtctgacacaatccctaatcaagttt
gataataataataatttatttcctcttatta
gtattgctgatgcgtgggccaccaatcaatc
gtaaagaaaaaaaatgttgagataaaaggtg
ggggtatcttctccttctctttttttttggc
taaaataaaagtggtttctggtagtctgaca
caatctctaatcgaaatatttatttttttct
cttagtattgctgatacgtgggccaccagcc
aataataaagaaaaaaaatgttagagataaa
aggcggagagtatcttccccttccttttttt
tggcgtaaatgaaagaaaagagaaaatctcc
cgtcgtctccttccttgcgccaagaaagacg
agccgcggctcaacaccggaggggaggggcg
ccgatctccatcgccaaggagagcagagcag
gggaggggatcctggtgagcctcctcttcct
gattcatctctctcccattctagcttcgggg
gactacttttgcctggaatttgctcgcgttc
gttcgtgcgttcgttcgttaaccctagcttc
ttctcttctagatctggaggaagctcttctc
ctccttaatttcagagccttaatacaagtag
taacagtttaacctcccccatgtcccaagtt
ggatccgcccctgcgagttccgatattgggt
cctcccaattctcaatgccattttgttcatc
ggggggcatatggttcatttttgcctgcatt
gattcaaatgtggtttcgaatcgtttgtgaa
attcgcgggtgtacttgtttatgatacatga
ggccttttttcccccatgaggaggcaaactt
tttagtgggtggatccactagttcatgcctc
aattttttttctcctcttttaagttttccaa
agagctacattgttgtaaagtgtctgataca
attgattgtttattcaggttagcgcttttgg
cgtgtgattgatttctaaacgaattttgggc
cgtgaggggaagttcaatcatggcagggtct
cttgccgcctcagcattcttcccaggtccag
gctcatctcctgcagca
NippFatB6 promoter
(SEQ ID NO: 54)
acagaaatttcgctggccatgcacataatct
tctctttgtcaaagagctggaatccaaaatg
attgctcgaagatttcgtgaagatagataga
accatcggctagcaaaggagaggaaataaaa
aaacaaaaaaaaagtttttttgtgggctcca
ccttgcgctgcactgagctgaccaaattgac
cataccgcacagagactgagggaggggcact
tccgtcatttcgtataagcgtatacgaatac
gtatctcatacgcgctctgtatatatagacg
gtaacggctccgcgtcgtgtgagttggcgag
cccgaggagcggaggcggccacaagtctaat
ccgcgtcgtctgcgcgttcgtgggcgaggag
gagaaagaagaggaggaagagagggaagggg
gcttgatttgatttgggcgcgtctcgtggag
tatccggtgagttcttggcgatctggcgagg
cgagtgatgagtgattcctgctgctgctggg
ggattttggcgtgattttcgttggttgcatt
ttgtttctttttttttgtatcgatttgttgg
agctttattcggtagatctggtcgattccat
ggtgagttgtatcggcgccggagtgatagct
gattctgtttttttgtgtgattttttttttg
ttttggaaatagggtttgtgtcgaattgagg
gcattttttttccttaggcaatgcaggattt
cgttttgtatgtttttgcgtggaatggatat
gaacagacctcgaacaaatggaagaatttgt
attttgtatgatggattgcaatgcgatactt
gttttggggcgtgattcgattgaaataaatg
aaatattagagttattttgggattcctgttt
gctgcgcctttttttttagcatttcttgata
tgaacaagagaagaagggctgaatttttttc
ttagctttggaggcatttactgtcccagtat
tttctcctaccggaagcagaatattttgttt
gattggagggttgcctccctttgccaaattg
aatcaaatgttctcggatgttttaaaatttc
cgtggactctttttgccccaggggagaccgc
ttttagcagctggatcccgtgttttcatttc
aagttcttgttttcctagtctccatatattt
ctgattgttaactcgtattctctacctcaca
tatgcaaaatcacacttgcgtcgttctgtaa
ttagttagattctgcaagaaaaatccggaat
tttcaagcatgctagtagttttaaattgatg
ccatgttttttagacaatgttaattgatgcc
atatgactataggacacattatattgcgttt
ctgaatataccacctcatgaaactcataatt
ttgttgattaattgttcaggttgccccttct
agtgtgtaacttggagcaaatttggaccctg
agacgcaaatcagtcatggctggttctcttg
cggcgtctgcattcttccctgtcccagggtc
ttcccctgcagcttcggctagaagctctaag
aacacaaccggtgaattgccag
NippFatB11 promoter
(SEQ ID NO: 55)
ttctcgtatcctagcccatatatttttacag
attcgggttcaagctcgcataatatcgggta
tccaattttctctggcatattcttcatgcaa
gccttgagttgagtgaacgatcgggaaatac
ctgccccattttcacccctaccagtgggtgg
gggcattgcatgaacgaaacatggaaaagga
atcgacttgagaggctagacctgctagctct
actgggtctggatggtcatgcatttatttga
aaaaaaaacacgccgcaagctcgataacccc
gacctcaacggcaaccatgacaacaatatgc
atgacattgggggaggagaaaagacatgttg
ataactagagggcgcggctacgcatgtaaat
gcggagagaaggaacgacggtagctcaaaaa
gtaagagcgtcggtggcggtggtgcgtggag
cgacactaaagttagtggttgctggtggtct
gacacaaatccctaatcgaaatatttatttt
ttctcttagtattactgatacgtgggccacc
cgccaattataaagaaaaatgttgagataaa
aggtggagtatctttcccttccttccctttt
ttgccttaaaaaaaagagcgtcggtgacggc
cgtgcgtgtagcaatactaaagttagtggtt
gatggtggtctgacacaatccctaatcgagt
ttgataataatatttatttttctcttagtat
cgctgatacgtgggccaccagccaatcataa
aggaaaaaaaatgttgagataaaaggtggat
agtatcttcccccttccttcccttttttggc
gtaaaagaaagaggagaaattctcccgtcgt
ctccttccttgcgccaagaaagacgagccgc
ggctcaacagcggagtggaggggcgccgatc
tccatcgccgaggagagcagagcaggggagg
ggaggggatcctggtgagcctcctcttcctg
attcacctctctctcattctagcttcggggg
actacttttgcctcgaatttgcttgcgttcg
ttcgttaaccctagcttcttctcttctagat
ctggaggaagctcttctcctccttaatttca
gagccttaatacaagtagtaacagtttaacc
ccccccccccccatgtcccaagttggatccg
cccctgcgagttccgatattgggtcctccca
attctcaatgccattttgttcatcggggggc
atatggttcattttgcctgcattgattcaaa
tgtggtttcgaatcgtttgggaaattcgcgg
gtgtacttgtttatgatatatgaggcctttt
ttttccccatgaggaggcaaactttttagtg
ggtggatccactagttcatgcctcaattttt
tttctcctcttttaagttttccaaagagcta
cattgttgtaaagtgtctaatacaattgatt
gtttattcaggttagcgcttttggcgtgtga
ttgatttctaaacgaattttgggccgtgaag
ggaagttcaatcatggcagggtctcttgccg
cctcagcattcttcccaggtccaggctcatc
tcctgcagca
OeFatB2 promoter
(SEQ ID NO: 56)
acagaaatttcgctggccatgcacataatct
tctctttgtcaaagagctggaatccaaaata
attgctcgaagatttcgtgtagatagaacca
tcggccagcaaaggagtggaaaaagaaaacg
tttttttgtgggccccaccggcgctgcactg
agctgaccaaatgactataccgcacagaggg
aggggggcatttccgtcctttcgtatagacg
tatatgaatacgtatctcatacgcgctctgt
gtatatagacgcacctgcgccagaggagacg
gtaacggctcagcgaaggggagagagagaag
aaggaaaaaaaaactcatctctctctctctc
tcttgtttctctctgcctcgcgtcgtgtgag
ttggcgagcccgaggagcggaggccacaagt
ctaatccgccgtatctaatccgctcgaccgc
gtctgcgcgtgcgtgggtgaggagaaagagg
aggaggtggaggagaaagagagggggcttga
tctgggcgcttctcgtggagtatccggtgag
ttcttggcgatctggcgaggcgagtggtgag
tggctccgcgtgtgctgctgccgggggattt
tggcgtgattttcgttggttgcattttgttt
tttttgtgtatcgatttgttggagcttattc
ggtagatctggtcgattacatggtgagttgt
ataggcgccagagtgatagctgattttgttt
tggtgtaaattttgttttggaaggagggttt
gtgtcgatttgagggcatttttcctcgggca
atgcaggatttggatttgtatgtttttgcat
ggaatggatatgaacggacctcaaacaaatg
gaggagtttgtactttggatggattgcaatg
tggttttgaggcgtgattcggttgaagaaat
gaactaaggaatattcgagttattttgggat
tcctgtttgctgcgcctttttttagcatttc
ttgatatgaacaagagaaaaagggctgattt
tttccttagctttggaggcatttactgtccc
agtattttctcctaccggaagcagaatattt
tgtttgattggagggttgtctccttttgcca
aatcgaatcaaatgctctcggatgttttgaa
atttcggtggactccttttgcccaagggagg
ccacttttagcagctgtggatcccgtgtttt
cattcaagttcttgttttcctagtctccata
tatttctgattattaactcggattctctaca
tcaaatatgcgaaatcacacttgcgtcgttc
tgtagttagttaggttctgcaagacaaatcc
gaaatttttaagcatgctgtcatagtatcat
tggattcccccttttactgggaagaaagttc
taccttttgtgctttcggtagtttttaattg
atgccatgttttttagataatgttaattgat
gccatgtgactataggacacattatattgcg
tttctgaatatatcacctcatgaaactcata
attttgttgattatttgttcaggttgcccct
tctagcgtgtagcttcgagcaaatttggact
ctgaggcgcatttcggtcatggctggttctc
ttgcagcgtctgcattcttccctagcccagg
gtcttcccctgcagcatcgactagaagttct
aagaatacaaccagtgaattgccagagaatt
tgag
OeFatB6 promoter
(SEQ ID NO: 57)
acagaaatttcgctggccatgcacaatcttc
tctttgtcaaagagctggaatccaaaatgat
tgctcgaagatttcgtgtagatagatagaac
catcggccagcaaaggagaggggaacaaaaa
ggaaaaaagtctttttgtgggccccacctgc
actgcactgggttgaccaaattgaccatacc
gctcagaggggggggggcatttccgtccttt
cgtataaacgtatacgaatacgtatctcaca
cgcgctctgtatatatagacggtaacggctc
cgcgaaggagagagaagaagaagaaaaaaaa
agtcatctttctctctcttgtttctctctgc
ctcgagtcgcggctgaacaggggaggggcgg
cgatctccatctggcgagcagagcagggaag
gggaggggatcctggtgagcatccacatcct
ttttctgattcatatatctctcccaccnggg
agtacttttgtctggaatttgcttgcattaa
ccctagcttctcttgtagatctggaagaagc
tcttctcttaatttcagagccttaaccttaa
tacaagtaacagtttgttgtttgttccccca
aaagtttgctgcgcgtttttttggcatctct
tgatatgaacaagagaaacaagctgaatttt
ttcttacctttggaagcatttaccgtcccag
tattttctcctaccggtagtagaatattttg
tttgattggaggcttgccttcttttgctaaa
tcgaatcaaatgctctcggatgtttttaaaa
tttcggtggactccttttgccccaagggagg
ccagttttagcagctggatcccgtgttttca
tttcaacttcttgttttccttgtctccatat
atttctgattgttaactcggattctctacct
caaatatgtaatatcacactttaagacaaat
ccggaattttaagcatgctatcatagtatca
ttagattcccccttttacagggaagaaaagt
tctacattttgtgctttcggtagcttttaat
tgatgccatgttttttagacaatgttaattg
atgccatgtgactatagggcacattatattg
cgtttctgaatatatcacctcatgaaactga
taattttgttgattatttgttcagtttgccc
ttctagtgtgtaacttcgagcaaatttggac
cctgaggcgcagttcagtcatggctggttct
cttgcagcgtctgcattcttccctggcccag
ggtcttcccctgcagcatcagctagaagttc
taagaacacaaccggtgaattgccagagaat
ttgag

An alignment of promoter sequences of OeFatB6 and NippFatB6 showed that the promoter sequences are indeed far different from each other in some regions. The differences in oil content between wild rice and Nipponbare are therefore postulated to be due to different expressions of FatB6 caused by their different promoters.

```
Majority
ACAGAAATTTCGCTGGCCATGCACAXXATCTTCTCTTTGTCAAAGAGCTGGAATCCAAAATGATTGCTCGAAGATTTCGT
---------+---------+---------+---------+---------+---------+---------+---------+
                  10 20 30 40 50 60 70 80
OeFatB6 promoter.seq
ACAGAAATTTCGCTGGCCATGCACA--ATCTTCTCTTTGTCAAAGAGCTGGAATCCAAAATGATTGCTCGAAGATTTCGT
NippFatB6 promoter.seq
ACAGAAATTTCGCTGGCCATGCACATAATCTTCTCTTTGTCAAAGAGCTGGAATCCAAAATGATTGCTCGAAGATTTCGT
Majority
GXAGATAGATAGAACCATCGGCXAGCAAAGGAGAGGXXAXXAAAAAXXXXXAAAAAAGTXTTTTTGTGGGCXCCACCTXG
---------+---------+---------+---------+---------+---------+---------+---------+
                  90 100 110 120 130 140 150 160
OeFatB6 promoter.seq
GTAGATAGATAGAACCATCGGCCAGCAAAGGAGAGGGGAACAAAAAG---GAAAAAAGTCTTTTTGTGGGCCCCACCT-G
NippFatB6 promoter.seq
GAAGATAGATAGAACCATCGGCTAGCAAAGGAGAGGAAATAAAAAAACAAAAAAAAAGTTTTTTTGTGGGCTCCACCTTG
Majority
CXCTGCACTGXGXTGACCAAATTGACCATACCGCXCAGAGXXXXXGGGXGGGGCAXTTCCGTCXTTTCGTATAAXCGTAT
---------+---------+---------+---------+---------+---------+---------+---------+
                  170 180 190 200 210 220 230 240
OeFatB6 promoter.seq
CACTGCACTGGGTTGACCAAATTGACCATACCGCTCAGAGG----GGGGGGGGCATTTCCGTCCTTTCGTATAAACGTAT
NippFatB6 promoter.seq
CGCTGCACTGAGCTGACCAAATTGACCATACCGCACAGAGACTGAGGGAGGGGCACTTCCGTCATTTCGTATAAGCGTAT
Majority
ACGAATACGTATCTCAXACGCGCTCTGTATATATAGACGGTAACGGCTCCGCGXXGXGXGAGXXXXXXXXXXXXAGXAGX
---------+---------+---------+---------+---------+---------+---------+---------+
                  250 260 270 280 290 300 310 320
OeFatB6 promoter.seq
ACGAATACGTATCTCACACGCGCTCTGTATATATAGACGGTAACGGCTCCGCGAAG-GAGAG------------AGAAGA
NippFatB6 promoter.seq
ACGAATACGTATCTCATACGCGCTCTGTATATATAGACGGTAACGGCTCCGCGTCGTGTGAGTTGGCGAGCCCGAGGAGC
Majority
XGAXGXXXXXAXAAGTCXXXTXXXXXTCXXXTGXXXXTXXXTGXXXXXXGXXGXGXXXGAAXAGGXGXXXXXXXXXXXAG
---------+---------+---------+---------+---------+---------+---------+---------+
                  330 340 350 360 370 380 390 400
OeFatB6 promoter.seq
AGAAGAAAAAAAAAGTCATCTTTCTCTCTCTTGTTTCTCTCTGCCTCGAGTCGCGGCTGAACAGGGG-----------AG
NippFatB6 promoter.seq
GGAGGCGGCCACAAGTCTAATCCGCGTCGTCTGCGCGTTCGTGGGCGAGGAGGAGAAAGAAGAGGAGGAAGAGAGGGAAG
Majority
GGGXXXXXXXXXXXXXXXXXXXXXXXXXXXXXXXXXXXCGGXGAXXTCXXXXXXATCTGGCGAGXXGAGXXXXGAXXGXX
---------+---------+---------+---------+---------+---------+---------+---------+
                  410 420 430 440 450 460 470 480
OeFatB6 promoter.seq
GGG-----------------------------------CGGCGATCTCC-----ATCTGGCGAGCAGAGCAGGGAAGGGG
NippFatB6 promoter.seq
GGGGCTTGATTTGATTTGGGCGCGTCTCGTGGAGTATCCGGTGAGTTCTTGGCGATCTGGCGAGGCGAGTGATGAGTGAT
Majority
XXXXXXXXXXXXXXGGGGATXXTGGXGXGXXXXXXXXXXXXXXCATXXXXXTXCTTTTTXTXXXXXXXGATTXXTXXXXX
---------+---------+---------+---------+---------+---------+---------+---------+
                  490 500 510 520 530 540 550 560
OeFatB6 promoter.seq
-------------AGGGGATCCTGGTGAG--------------CATCCACATCCTTTTTCT-------GATTCAT-----
NippFatB6 promoter.seq
TCCTGCTGCTGCTGGGGGATTTTGGCGTGATTTTCGTTGGTTGCATTTTGTTTCTTTTTTTTTGTATCGATTTGTTGGAG
Majority
XXXXXXXXXXXAXATCTXXXXXXXTCCXXXXXXXXXXXXXXXXXCXCCXGXGXGXXXXXXXXXXXTXXTTTTXTXTGXXA
---------+---------+---------+---------+---------+---------+---------+---------+
                  570 580 590 600 610 620 630 640
OeFatB6 promoter.seq
-----------ATATCT------CTCC-----------------CACCNGGGAG-----------TACTTTTGTCTGGAA
NippFatB6 promoter.seq
CTTTATTCGGTAGATCTGGTCGATTCCATGGTGAGTTGTATCGGCGCCGGAGTGATAGCTGATTCTGTTTTTTTGTGTGA
Majority
TTTXXTTXXXGXXTTXXXXXTAGXXTXTXTXXXXXATXXXGXXXAXXXTXTTXXCTTAXXXAXTXCAGXXXXTXXXXXXX
---------+---------+---------+---------+---------+---------+---------+---------+
                  650 660 670 680 690 700 710 720
OeFatB6 promoter.seq
TTTGCTT---GCATTAACCCTAGCTTCTCTTGTAGATCTGGAAGAAGCTCTTCTCTTA---ATTTCAGAGCCTTA-----
NippFatB6 promoter.seq
TTTTTTTTTTGTTTTGGAAATAGGGTTTGTGTCGAATTGAGGGCATTTTTTTTCCTTAGGCAATGCAGGATTTCGTTTTG
Majority
XXXXXXXXXXXXXXXXXXXXXXXXXXXXXXACCTXXAAXAXAXGXAAXAXTTTGTXXTTTGTXXXXXXXXXXXXXXXXXX
---------+---------+---------+---------+---------+---------+---------+---------+
                  730 740 750 760 770 780 790 800
OeFatB6 promoter.seq
------------------------------ACCTT-AATACAAGTAACAGTTTGTTGTTTGT------------------
NippFatB6 promoter.seq
TATGTTTTTGCGTGGAATGGATATGAACAGACCTCGAACAAATGGAAGAATTTGTATTTTGTATGATGGATTGCAATGCG
Majority
XXXXXXXXXXXXXXXXXXXXXTCXXXXXAAAXXXXXXXXXXXXXXXXXXXXXXXXXXXXXXXXXGTTTGCTGCGCXTTTT
---------+---------+---------+---------+---------+---------+---------+---------+
                  810 820 830 840 850 860 870 880
OeFatB6 promoter.seq
---------------------TCCCCCAAAA---------------------------------GTTTGCTGCGCGTTTT
NippFatB6 promoter.seq
ATACTTGTTTTGGGGCGTGATTCGATTGAAATAAATGAAATATTAGAGTTATTTTGGGATTCCTGTTTGCTGCGCCTTTT
Majority
TTTXXXGCATXTCTTGATATGAACAAGAGAAXXAXGXCTGAATTTTTTXCTTAXCTTTGGAXGCATTTACXGTCCCAGTA
---------+---------+---------+---------+---------+---------+---------+---------+
                  890 900 910 920 930 940 950 960
OeFatB6 promoter.seq
TTT--GGCATCTCTTGATATGAACAAGAGAAACAAG-CTGAATTTTTT-CTTACCTTTGGAAGCATTTACCGTCCCAGTA
NippFatB6 promoter.seq
TTTTTAGCATTTCTTGATATGAACAAGAGAAGAAGGGCTGAATTTTTTTCTTAGCTTTGGAGGCATTTACTGTCCCAGTA
Majority
TTTTCTCCTACCGGXAGXAGAATATTTTGTTTGATTGGAGGXTTGCCTXCXTTTGCXAAATXGAATCAAATGXTCTCGGA
---------+---------+---------+---------+---------+---------+---------+---------+
                  970 980 990 1000 1010 1020 1030 1040
OeFatB6 promoter.seq
TTTTCTCCTACCGGTAGTAGAATATTTTGTTTGATTGGAGGCTTGCCTTCTTTTGCTAAATCGAATCAAATGCTCTCGGA
NippFatB6 promoter.seq
TTTTCTCCTACCGGAAGCAGAATATTTTGTTTGATTGGAGGGTTGCCTCCCTTTGCCAAATTGAATCAAATGTTCTCGGA
Majority
TGTTTTXAAAATTTCXGTGGACTCXTTTTGCCCCAXGGGAGXCCXXTTTTAGCAGCTGGATCCCGTGTTTTCATTTCAAX
---------+---------+---------+---------+---------+---------+---------+---------+
                  1050 1060 1070 1080 1090 1100 1110 1120
OeFatB6 promoter.seq
TGTTTTTAAAATTTCGGTGGACTCCTTTTGCCCCAAGGGAGGCCAGTTTTAGCAGCTGGATCCCGTGTTTTCATTTCAAC
NippFatB6 promoter.seq
TGTTTT-AAAATTTCCGTGGACTCTTTTTGCCCCAGGGGAGACCGCTTTTAGCAGCTGGATCCCGTGTTTTCATTTCAAG
Majority
TTCTTGTTTTCCTXGTCTCCATATATTTCTGATTGTTAACTCGXATTCTCTACCTCAXATATGXAAXATCACACTTXXXX
---------+---------+---------+---------+---------+---------+---------+---------+
                  1130 1140 1150 1160 1170 1180 1190 1200
OeFatB6 promoter.seq
TTCTTGTTTTCCTTGTCTCCATATATTTCTGATTGTTAACTCGGATTCTCTACCTCAAATATGTAATATCACACTTTAAG
NippFatB6 promoter.seq
TTCTTGTTTTCCTAGTCTCCATATATTTCTGATTGTTAACTCGTATTCTCTACCTCACATATGCAAAATCACACTT
Majority
XXXXXXXXXXXXXXXXXXGCXTXXTXXXXTAXTXTXXTTAGATTCXXCXXXXXXXXXXXAAGAAAAXTXCXXXATTTTXX
---------+---------+---------+---------+---------+---------+---------+---------+
                  1210 1220 1230 1240 1250 1260 1270 1280
OeFatB6 promoter.seq
ACAAATCCGGAATTTTAAGCATGCTATCATAGTATCATTAGATTCCCCCTTTTACAGGGAAGAAAAGTTCTACATTTT-G
NippFatB6 promoter.seq
------------------GCGTCGTTCTGTAAT-TAGTTAGATTCTGC-----------AAGAAAAATCCGGAATTTTCA
Majority
XGCXTXCXXGTAGXTTTXAATTGATGCCATGTTTTTTAGACAATGTTAATTGATGCCATXTGACTATAGGXCACATTATA
---------+---------+---------+---------+---------+---------+---------+---------+
                  1290 1300 1310 1320 1330 1340 1350 1360
OeFatB6 promoter.seq
TGCTTTC-GGTAGCTTTTAATTGATGCCATGTTTTTTAGACAATGTTAATTGATGCCATGTGACTATAGGGCACATTATA
NippFatB6 promoter.seq
AGCATGCTAGTAGTTTTAAATTGATGCCATGTTTTTTAGACAATGTTAATTGATGCCATATGACTATAGGACACATTATA
Majority
TTGCGTTTCTGAATATAXCACCTCATGAAACTXATAATTTTGTTGATTAXTTGTTCAGXTTGCCCXTTCTAGTGTGTAAC
---------+---------+---------+---------+---------+---------+---------+---------+
                  1370 1380 1390 1400 1410 1420 1430 1440
OeFatB6 promoter.seq
TTGCGTTTCTGAATATATCACCTCATGAAACTGATAATTTTGTTGATTATTTGTTCAGTTTGCCC-TTCTAGTGTGTAAC
NippFatB6 promoter.seq
TTGCGTTTCTGAATATACCACCTCATGAAACTCATAATTTTGTTGATTAATTGTTCAGGTTGCCCCTTCTAGTGTGTAAC
Majority
TTXGAGCAAATTTGGACCCTGAGXCGCAXXTCAGTC
---------+---------+---------+------
                  1450 1460 1470
OeFatB6 promoter.seq
TTCGAGCAAATTTGGACCCTGAGGCGCAGTTCAGTC
                                                                      1197
NippFatB6 promoter.seq
TTGGAGCAAATTTGGACCCTGAGACGCAAATCAGTC
                                                                      1441
```

The consensus FatB6 promoter sequence shown above is found in SEQ ID NO: 69 (without any nucleotide gaps).

Rice FatB6 Confers Resistance Against Rice Brown Planthopper and Rice Blast Fungus

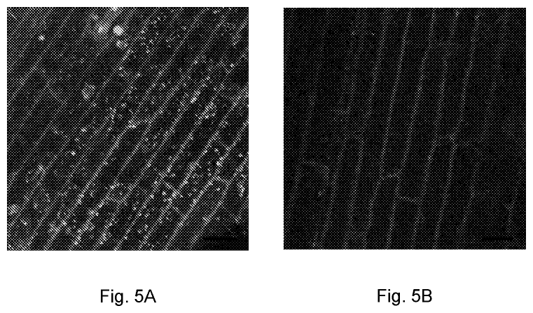

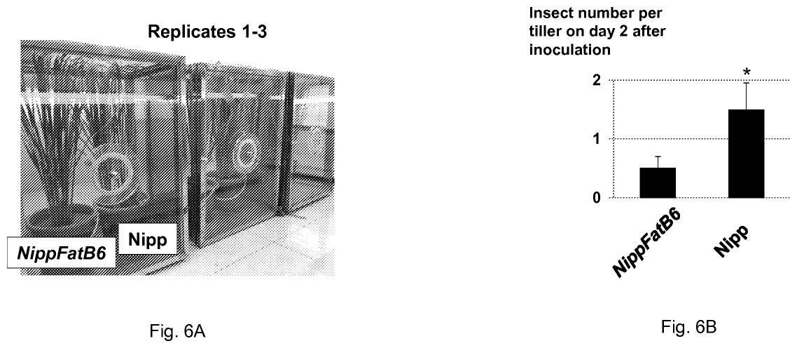

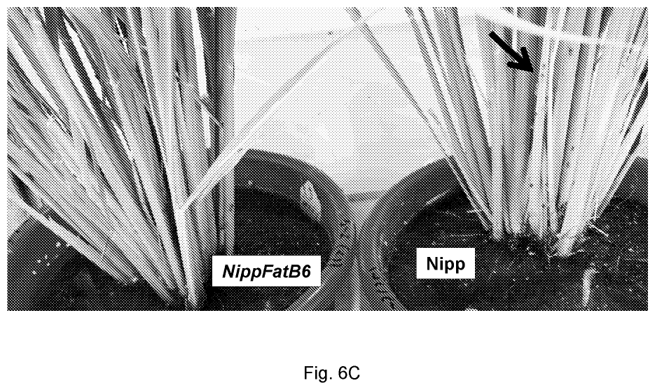

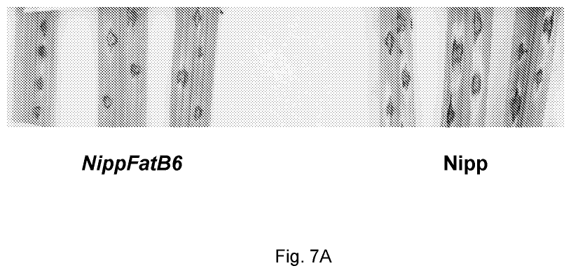

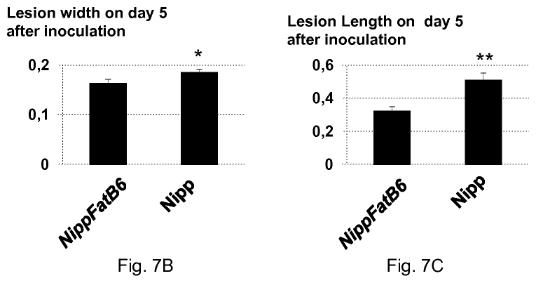

Wild rice possesses resistance against most of the insect pests and diseases including the major pest, rice brown planthopper, and the disease rice blast fungus (Fu et al. 2007). It was hypothesized that the high oil content caused by FatB6 in wild rice may confer significantly to the resistance. To demonstrate the hypothesis, the FatB genes were overexpressed in the Nipponbare background using a strong promoter, barley SBEIIb promoter (Su et al. 2015) to test how efficiently the different genes can increase oil content in Nipponabre rice and in consequence lead to resistance against to the pest and disease. The first available transformant was a rice line with overexpression of NippFatB6, see FIGS. 5A and 5B. When the oil abundance was observed in the transformant, the oil abundance was much higher in leaf sheath than in the control, see FIGS. 5A and 5B. The same rice was used to test resistance against rice brown planthopper and rice blast fungus and all three biological replicates showed significant resistance against the pest, see FIGS. 6A to 6C, and the disease, see FIGS. 7A to 7C.

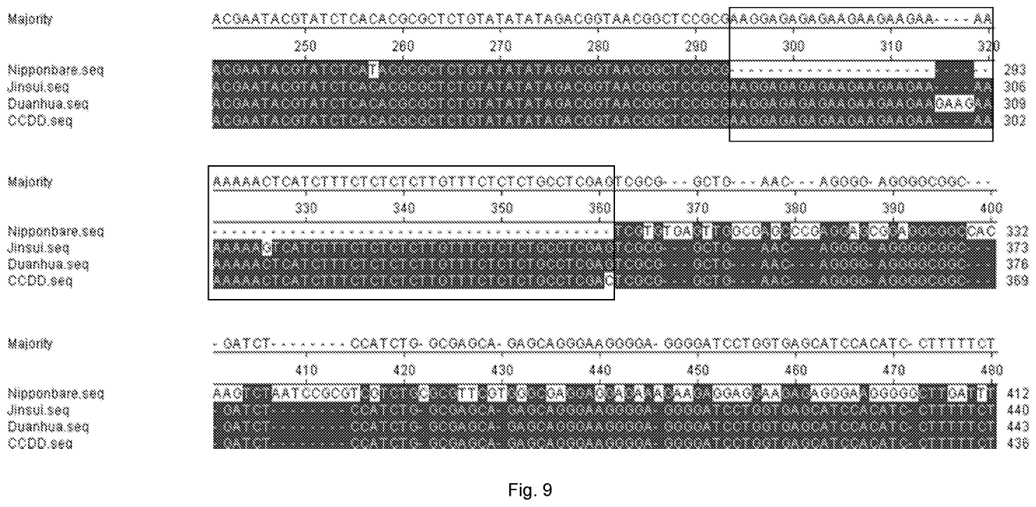

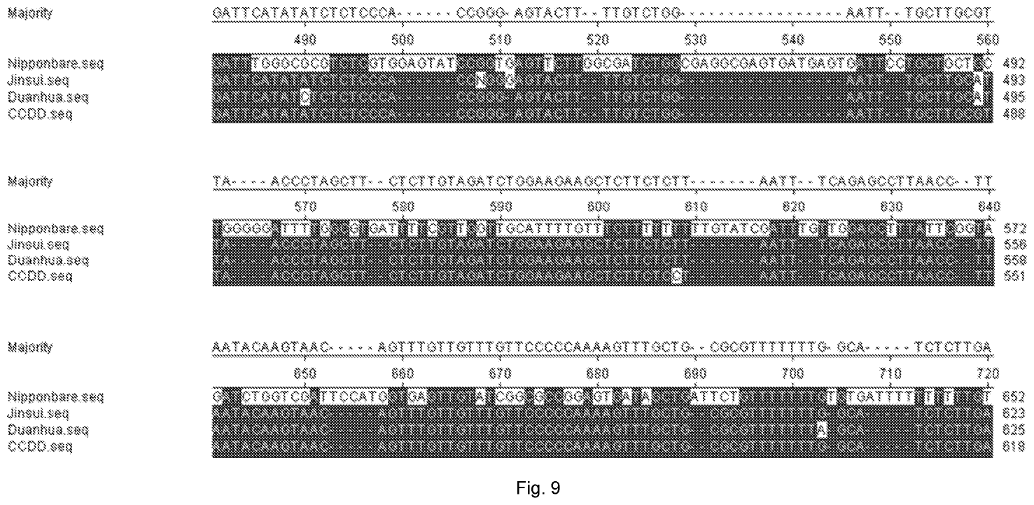

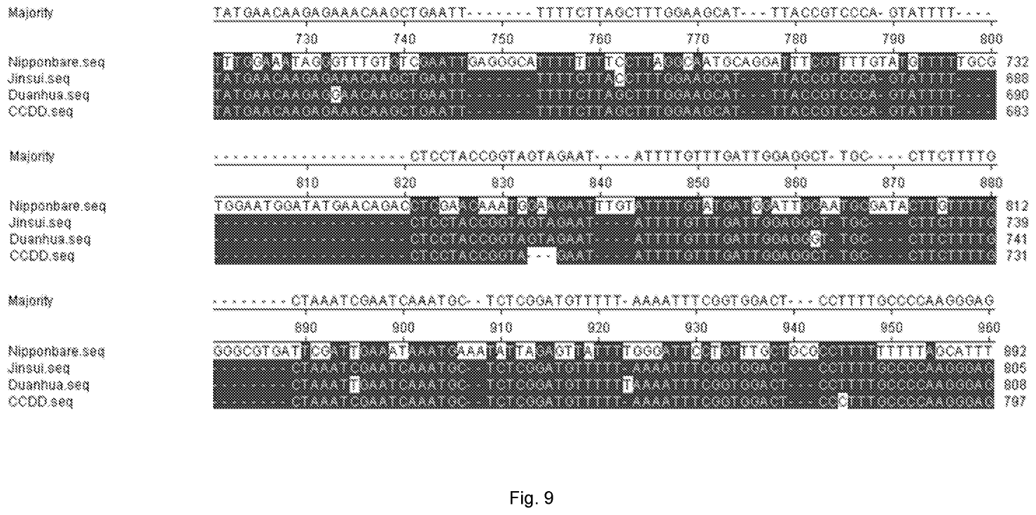

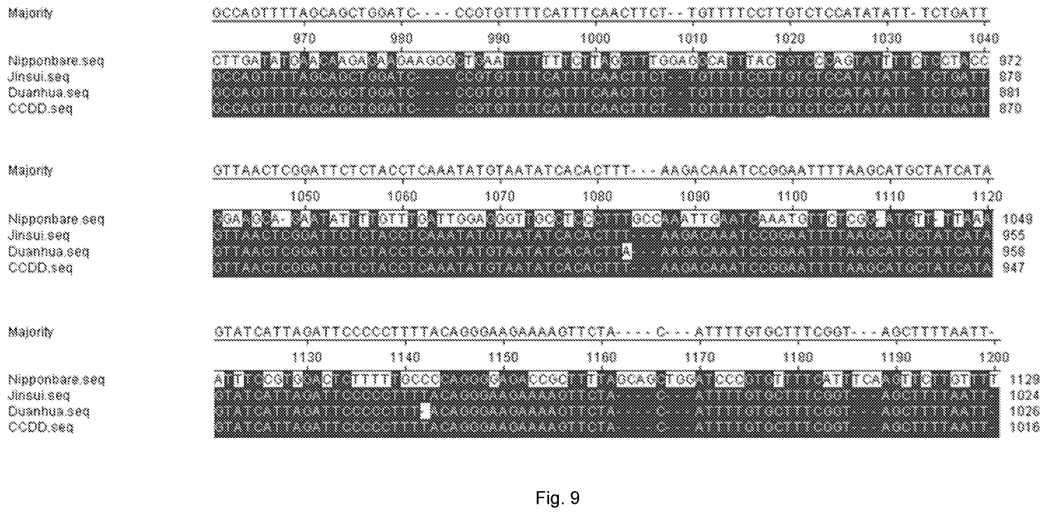

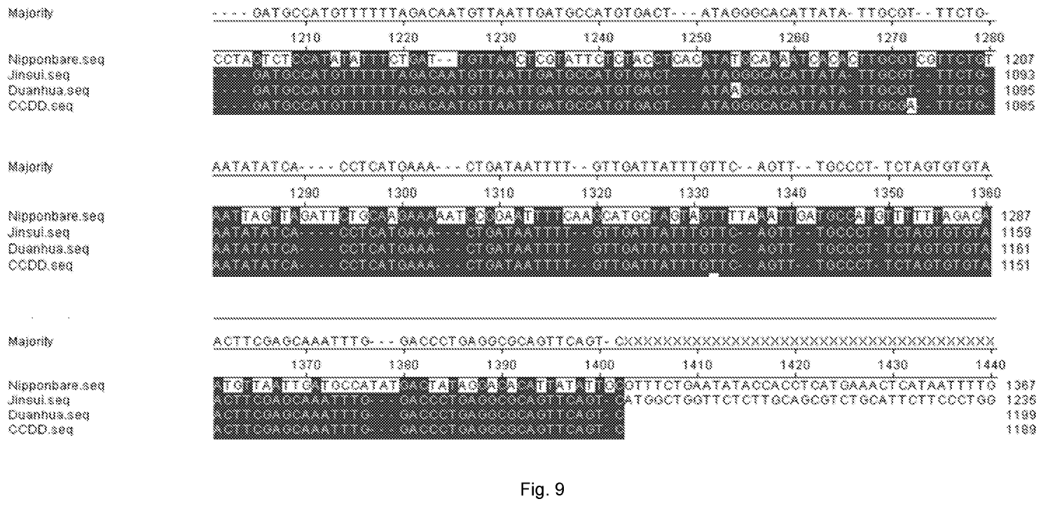

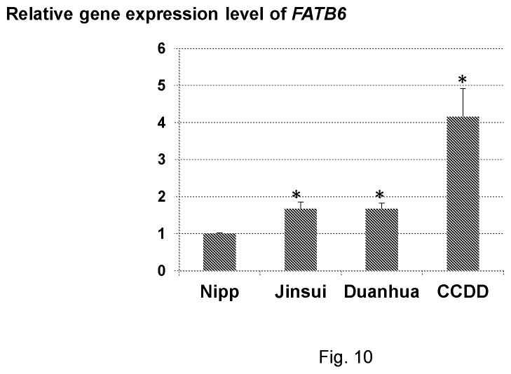

Interestingly, when the promoter regions of FatB6 were isolated from two additional wild rice, Duanhua (Oryza brachyantha) and CCDD (Oryza latifolia), and aligned with the FatB6 promoter regions of Nipponbare and Jinsui (Oryza eichingen), it was found that all three wild rice possess a nucleotide sequence with CT-rich motifs similar to the CT-rich motifs in the 35S promoter (Pauli et al. 2004), but not in Nipponbare, see FIG. 9. The CT-rich motifs may play a role in high expression of FatB6 in wild rice. FIG. 10 illustrates an analysis of FatB6 gene expression in wild rice and Nipponbare, which supports the notion.

O. brachyantha FatB6 Promoter (SEQ ID NO: 66)

ACAGAAATTTCGCTGGCCATGCACAATCTTCTCTTT
GTCAAGGAGCTGGAATCCAAAATGATTGCTCGAAG
ATTTCGTGTAGATAGATAGAACCATCGGCCAGCAA
AGGAGAGGGGAAAAAAAAAATGAAAAACGTCTTTT
TGTGGGCCCCACCTGCACTGCACTGAGTTGACCAA
GTTGACCATACCGCTCAGAGGGGGGGCATTTCCGT
CCTTTCGTATAAACGTATACGAATACGTATCTCAC
ACGCGCTCTGTATATATAGACGGTAACGGCTCCGC
GAAGGAGAGAGAAGAAGAAGAAGAAGAAAAAAACT
CATCTTTCTCTCTCTTGTTTCTCTCTGCCTCGAGT
CGCGGCTGAACAGGGGAGGGGCGGCGATCTCCATC
TGGCGAGCAGAGCAGGGAAGGGGAGGGGATCCTGG
TGAGCATCCACATCCTTTTTCTGATTCATATCTCT
CTCCCACCGGGAGTACTTTTGTCTGGAATTTGCTT
GCATTAACCCTAGCTTCTCTTGTAGATCTGGAAGA
AGCTCTTCTCTTAATTTCAGAGCCTTAACCTTAAT
ACAAGTAACAGTTTGTTGTTTGTTCCCCCAAAAGT
TTGCTGCGCGTTTTTTTAGCATCTCTTGATATGAA
CAAGAGGAACAAGCTGAATTTTTTCTTAGCTTTGG
AAGCATTTACCGTCCCAGTATTTTCTCCTACCGGT
AGTAGAATATTTTGTTTGATTGGAGGGTTGCCTTC
TTTTGCTAAATTGAATCAAATGCTCTCGGATGTTT
TTTAAAATTTCGGTGGACTCCTTTTGCCCCAAGGG
AGGCCAGTTTTAGCAGCTGGATCCCGTGTTTTCAT
TTCAACTTCTTGTTTTCCTTGTCTCCATATATTTC
TGATTGTTAACTCGGATTCTCTACCTCAAATATGT
AATATCACACTTAAAGACAAATCCGGAATTTTAAG
CATGCTATCATAGTATCATTAGATTCCCCCTTTAC
AGGGAAGAAAAGTTCTACATTTTGTGCTTTCGGTA
GCTTTTAATTGATGCCATGTTTTTTAGACAATGTT
AATTGATGCCATGTGACTATAAGGCACATTATATT
GCGTTTCTGAATATATCACCTCATGAAACTGATAA
TTTTGTTGATTATTTGTTCAGTTTGCCCTTCTAGT
GTGTAACTTCGAGCAAATTTGGACCCTGAGGCGCA
GTTCAGTC

O. latifolia FatB6 Promoter (SEQ ID NO: 67)

ACAGAAATTTCGCTGGCCATGCACAATCTTC
TCTTTGTCAAAGAGCTGGAATCCAAAATGA
TTGCTCGAAGATTTCGTGTAGATAGATAGA
ACCATCGGCCAGCAAAGGAGAGGGGAACAA
AAAGGAAAAAAGTCTTTTTGTGGGCCCCAC
CTGCACTGCACTGAGTTGACCAAATTGACC
ATACCGCTCAGAGGGGGGCATTTCCGTCCT
TTCGTATAAACGTATACGAATACGTATCTC
ACACGCGCTCTGTATATATAGACGGTAACG
GCTCCGCGAAGGAGAGAGAAGAAGAAGAAA
AAAAAACTCATCTTTCTCTCTCTTGTTTCT
CTCTGCCTCGACTCGCGGCTGAACAGGGGA
GGGGCGGCGATCTCCATCTGGCGAGCAGAG
CAGGGAAGGGGAGGGGATCCTGGTGAGCAT
CCACATCCTTTTTCTGATTCATATATCTCT
CCCACCGGGAGTACTTTTGTCTGGAATTTG
CTTGCGTTAACCCTAGCTTCTCTTGTAGAT
CTGGAAGAAGCTCTTCTCCTAATTTCAGAG
CCTTAACCTTAATACAAGTAACAGTTTGTT
GTTTGTTCCCCCAAAAGTTTGCTGCGCGTT
TTTTTGGCATCTCTTGATATGAACAAGAGA
AACAAGCTGAATTTTTTCTTAGCTTTGGAA
GCATTTACCGTCCCAGTATTTTCTCCTACC
GGTAGAATATTTTGTTTGATTGGAGGCTTG
CCTTCTTTTGCTAAATCGAATCAAATGCTC
TCGGATGTTTTTAAAATTTCGGTGGACTCC
CTTTGCCCCAAGGGAGGCCAGTTTTAGCAG
CTGGATCCCGTGTTTTCATTTCAACTTCTT
GTTTTCCTTGTCTCCATATATTTCTGATTG
TTAACTCGGATTCTCTACCTCAAATATGTA
ATATCACACTTTAAGACAAATCCGGAATTT
TAAGCATGCTATCATAGTATCATTAGATTC
CCCCTTTTACAGGGAAGAAAAGTTCTACAT
TTTGTGCTTTCGGTAGCTTTTAATTGATGC
CATGTTTTTTAGACAATGTTAATTGATGCC
ATGTGACTATAGGGCACATTATATTGCGAT
TCTGAATATATCACCTCATGAAACTGATAA
TTTTGTTGATTATTTGTTCAGTTTGCCCTT
CTAGTGTGTAACTTCGAGCAAATTTGGACC
CTGAGGCGCAGTTCAGTC

The consensus FatB6 promoter sequence shown in FIG. 9 is found in SEQ ID NO: 68 (without any nucleotide gaps).

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible.

### REFERENCES

Aslan, S., Hofvander, P., Dutta, P., Sitbon, F. & Sun, C. Transient silencing of the KASII genes is feasible in Nicotiana benthamiana for metabolic engineering of wax ester composition. Sci. Rep. 5, 11213 (2015).
Du, B., Zhang, W., Liu, B., Hu, J., Wei, Z., Shi, Z., He, R., Zhu, L., Chen, R., Han, B. & He G. Identification and characterization of Bph14, a gene conferring resistance to brown planthopper in rice. Proc. Natl. Acad. Sci. USA 106, 22163-22168 (2009).
Fu, X.-L., Lu, Y.-G., Liu X.-D. & Li, J.-Q. Progress on transferring elite genes from non-AA genome wild rice into Oryza sativa through interspecific hybridization. Chinese J. Rice Sci. 21, 559-566 (2007).
Hiei, Y., Ohta, S., Komari, T. & Kumashiro, T. Efficient transformation of rice (Oryza sativa L.) mediated by Agrobacterium and sequence analysis of the boundaries of the T-DNA. Plant J. 6, 271-82 (1994).
Jain, M., Nijhawan, A., Tyagi, A. K. & Khurana, J. P. Validation of housekeeping genes as internal control for studying gene expression in rice by quantitative real-time PCR. Biochem. Biophys. Res. Commun. 345, 646-651 (2006).
Jin, Y., Fei, M., Rosenquist, S., Jin, L., Gohil, S., Sandström, C., Olsson, H., Persson, C., Hoglund, A.-S., Fransson, G., Ruan, Y., Åman, P., Jansson, C., Liu, C., Andersson, R. & Sun, C. A dual-promoter Gene orchestrates the sucrose-coordinated synthesis of starch and fructan in barley. Mol. Plant 10, 1556-1570 as a cover image paper (2017a).
Jin, Y., Hu, J., Liu, X., Ruan, Y., Sun, C. & Liu, C. T-6b allocates more assimilation product for oil synthesis and less for polysaccharide synthesis during the seed development of Arabidopsis thaliana. Biotech. Biofuels 10, 19 (2017b).
Li, Y., Yan, X., Wang, H., Liang, S., Ma, W.-B., Fang, M.-Y., Talbot, N.-J. & Wang Z.-Y. MoRic8 Is a novel component of G-protein signaling during plant infection by the rice blast fungus Magnaporthe oryzae. Mol. Plant Microbe Interact. 23, 317-331 (2010).
Nalawade, S., Nalawade, S., Liu, C., Jansson, C. & Sun, C. Development of an efficient tissue culture after crossing (TCC) system for transgenic improvement of barley as a bioenergy crop. Appl. Energy 91, 405-411 (2012).
Pauli, S., Rothnie, H.-M., Chen, G., Xiaoyuan He, X., & Hohn, T. The Cauliflower Mosaic Virus 35S Promoter Extends into the Transcribed Region. Journal of Virology 78, 12120-12128 (2004. Sun, C., Sathish, P., Ahlandsberg, S., Deiber, A. & Jansson, C. The two genes encoding starch-branching enzymes IIa and IIb are differentially expressed in barley. Plant Physiol. 118, 37-49 (1998).
Sun, C., Palmqvist, S., Olsson, H., Boren, M., Ahlandsberg, S. & Jansson, C. A novel WRKY transcription factor, SUSIBA2, participates in sugar signaling in barley by binding to the sugar-responsive elements of the isol promoter. Plant Cell 15, 2076-2092 (2003).
Sun, C., Hoglund, A.-S., Olsson, H., Mangelsen, E. & Jansson, C. Antisense oligodeoxynucleotide inhibition as a potent strategy in plant biology: identification of SUSIBA2 as a transcriptional activator in plant sugar signaling. Plant J. 44, 128-138 (2005).
Su, J., Hu, C., Yan, X., Jin, Y., Chen, Z., Guan, Q., Wang, Y., Zhong, D., Jansson, C., Wang, F., Schnürer, A. & Sun, C. Expression of barley SUSIBA2 transcription factor yields high-starch low-methane rice. Nature 523, 602-606 and as a featured paper (2015).
Zhang, M.-Z., Fang, J.-H., Yan, X., Liu, J., Bao, J.-S., Fransson, G., Andersson, R., Jansson, C., Aman, P. & Sun, C. Molecular insights into how a deficiency of amylose affects carbon allocation-carbohydrate and oil analysis and gene expression profiling in the seeds of a rice waxy mutant. BMC Plant Biol. 12, 230 (2012).

1. A rice plant material, wherein the rice plant material exhibits overexpression of a FatB gene selected from the group consisting of FatB2, FatB6, FatB11 and a combination thereof.

2. A rice plant material, having a FatB gene adapted for overexpression of a FatB enzyme selected from the group consisting of FatB2 as defined in SEQ ID NO: 42 or 48, FatB6 as defined in SEQ ID NO: 44 or 50, FatB11 as defined in SEQ ID NO: 46 or 52, a FatB enzyme having at least 80% sequence identify with a FatB enzyme as defined in SEQ ID NO: 42, 44, 46, 48, 50 or 52, and a combination thereof.

3. (canceled)

4. The rice plant material according to claim 1, wherein the rice plant material has higher oil and/or triacylglycerol (TAG) content as compared to a wild-type rice plant material lacking overexpression of the FatB gene or of the FatB enzyme.

5. The rice plant material according to claim 4, wherein the rice plant material has higher oil and/or TAG content in leaves, leaf sheath and/or stems as compared to the wild-type rice plant material.

6. (canceled)

7. The rice plant material according to claim 1, wherein the FatB gene is FatB6.

8. The rice plant material according to claim 1, wherein the FatB gene is an Oryza FatB gene.

9. The rice plant material according to claim 8, wherein the Oryza FatB gene is selected from the group consisting of an O. sativa FatB gene, an O. glaberrima FatB gene, an O. eichigeri FatB gene, an O. brachyantha FatB gene, an O. latifolia FatB gene and a combination thereof.

10. The rice plant material according to claim 9, wherein the Oryza FatB gene is an O. sativa FatB gene.

11. The rice plant material according to claim 10, wherein O. sativa FatB gene is selected from the group consisting of an O. sativa FatB2 gene as defined in SEQ ID NO: 41, an O. sativa FatB6 gene as defined in SEQ ID NO: 43, an O. sativa FatB11 gene as defined in SEQ ID NO: 45, and a combination thereof.

12. The rice plant material according to claim 1, wherein a promoter of the FatB gene, or at least a portion thereof, is replaced by a promoter selected from the group consisting of an ARP1 promoter, an H3F3 promoter, an HSP promoter, an H2BF3 promoter, a Cauliflower Mosaic Virus (CaMV) 35S promoter, a barley SBEIIb promoter and a heterologous FatB promoter.

13. The rice plant material according to claim 12, wherein the promoter of the FatB gene is replaced by the barley SBEIIb promoter.

14. The rice plant material according to claim 1, wherein a promoter of the FatB gene is an Oryza sativa FatB promoter or an O. glaberrima FatB promoter comprising a CT-rich motif.

15. The rice plant material according to claim 14, wherein the CT-rich motif is selected from the group consisting of:
(SEQ ID NO: 61)
AAGGAGAGAGAAGAAGAAGAAAAAAAAACT
CATCTTTCTCTCTCTTGTTTCTCTCTGCCT
CGAG;
(SEQ ID NO: 62)
AAGGAGAGAGAAGAAGAAGAAAAAAAAAGT
CATCTTTCTCTCTCTTGTTTCTCTCTGCCT
CGAG;
(SEQ ID NO: 63)
AAGGAGAGAGAAGAAGAAGAAGAAGAAAAA
AACTCATCTTTCTCTCTCTTGTTTCTCTCT
GCCTCGAG;
(SEQ ID NO: 64)
AAGGAGAGAGAAGAAGAAGAAAAAAAAACT
CATCTTTCTCTCTCTTGTTTCTCTCTGCCT
CGAC;
(SEQ ID NO: 65)
ACCAATCTCTCTCTACAAATCTATCTCTCT
CTATAA;
a combination thereof.

16. The rice plant material according to claim 1, having multiple copies of an endogenous FatB gene.

17. The rice plant material according to claim 1, having at least one copy of an endogenous FatB gene and at least one copy of a heterologous FatB gene.

18. The rice plant material according to claim 1, wherein the rice plant material is an Oryza sativa plant material or an O. glaberrima plant material.

19. The rice plant material according to claim 18, wherein the rice plant material is an O. sativa plant material.

20. The rice plant material according to claim 18 or 19, wherein a promoter of the FatB gene, or at least a portion thereof, is replaced by a heterologous FatB promoter selected from the group consisting of an O. eichigeri FatB promoter, an O. brachyantha FatB promoter, an O. latifolia FatB promoter, and a combination thereof.

21. The rice plant material according to claim 20, wherein the promoter of the FatB gene is replaced by an O. eichigeri FatB promoter selected from the group consisting of an O. eichigeri FatB2 promoter, an O. eichigeri FatB6 promoter and an O. eichigeri FatB11 promoter.

22. The rice plant material according to claim 21, wherein the O. eichigeri FatB promoter is selected from the group consisting of the O. eichigeri FatB2 promoter as defined in SEQ ID NO: 56 and the O. eichigeri FatB6 promoter as defined in SEQ ID NO: 57.

23. The rice plant material according to claim 20, wherein the promoter of the FatB gene is replaced by an O. eichigeri FatB6 promoter, an O. brachyantha FatB6 promoter, an O. latifolia FatB6 promoter, and a combination thereof.

24. The rice plant material according to claim 23, wherein the O. eichigeri FatB6 promoter is defined in SEQ ID NO: 57, the O. brachyantha FatB6 promoter is defined in SEQ ID NO: 66 and the O. latifolia FatB6 promoter is defined in SEQ ID NO: 67.

25. The rice plant material according to claim 18, wherein the FatB gene is a heterologous FatB gene.

26. The rice plant material according to claim 25, wherein the heterologous FatB gene is selected from the group consisting of an O. eichigeri FatB gene, an O. brachyantha FatB gene, an O. latifolia FatB gene, and a combination thereof.

27. The rice plant material according to claim 26, wherein the O. eichigeri FatB gene is selected from the group consisting of an O. eichigeri FatB2 gene, an O. eichigeri FatB6 gene, an O. eichigeri FatB11 gene, and a combination thereof.

28. The rice plant material according to claim 27, wherein the O. eichigeri FatB gene is selected from the group consisting of the O. eichigeri FatB2 gene as defined in SEQ ID NO: 47, the O. eichigeri FatB6 gene as defined in SEQ ID NO: 49, the O. eichigeri FatB11 gene as defined in SEQ ID NO: 51, and a combination thereof.

29. The rice plant material according to claim 27, wherein the O. eichigeri FatB gene is the O. eichigeri FatB6 gene.

30. The rice plant material according to claim 1, having a genomic nucleotide sequence encoding a sugar signaling in barley 2-like (SUSIBA2) transcription factor under transcriptional control of a promoter active in the rice plant material, wherein the genomic sequence encoding the SUSIBA2 transcription factor lacks at least a portion of an activation region of a sugar signaling in barley 1-like (SUSIBA1) promoter present in an intron of a wild-type version of the genomic nucleotide sequence encoding the SUSIBA2 transcription factor.

31. The rice plant material according to claim 30, wherein the genomic nucleotide sequence encoding the SUSIBA2 transcription factor lacks the activation region of the SUSIBA1 promoter.

32. The rice plant material according to claim 30, wherein the activation region of the SUSIBA1 promoter is as defined in SEQ ID NO: 58.

33. The rice plant material according to claim 30, wherein the genomic nucleotide sequence encoding the SUSIBA2 transcription factor lacks at least a portion of a sugar repressive region of the SUSIBA1 promoter.

34. The rice plant material according to claim 33, wherein the sugar repressive region of the SUSIBA1 promoter is as defined in SEQ ID NO: 59.

35. The rice plant material according to claim 33, wherein the genomic nucleotide sequence encoding the SUSIBA2 transcription factor lacks at least a portion of intron 2 comprising the activation region and the sugar repressive region of the SUSIBA1 promoter.

36. The rice plant material according to claim 30, wherein the SUSIBA1 promoter is as defined in SEQ ID NO: 60.

37. The rice plant material according to claim 30, wherein the genomic nucleotide sequence encoding the SUSIBA2 transcription factor is a genomic endogenous nucleotide sequence present in a chromosome of the rice plant material.

38. The rice plant material according to claim 1, wherein the rice plant material is selected from the group consisting of a rice plant, a rice plant cell, a rice tissue and a rice seed.

39. A method of improving resistance of a rice plant material against a biotic stress, the method comprising overexpressing a FatB gene in the rice plant material.

40. The method according to claim 39, wherein overexpressing the FatB gene comprises replacing a promoter of the FatB gene, or at least a portion thereof, by a promoter selected from the group consisting of an ARP1 promoter, an H3F3 promoter, an HSP promoter, an H2BF3 promoter, a Cauliflower Mosaic Virus (CaMV) 35S promoter, a barley SBEIIb promoter and a heterologous FatB promoter.

41. The method according to claim 40, wherein
the rice plant material is an Oryza sativa plant material or an O. glaberrima plant material; and
overexpressing the FatB gene comprises replacing a promoter of an O. sativa or O. glaberrima FatB gene, or at least a portion thereof, by a heterologous FatB promoter selected from the group consisting of an O. eichigeri FatB promoter, an O. brachyantha FatB6 promoter, an O. latifolia FatB6 promoter, and a combination thereof.

42. The method according to claim 39 or 110, wherein
the rice plant material is an Oryza sativa plant material or an O. glaberrima plant material; and
overexpressing the FatB gene comprises introducing a CT-rich motif into a promoter of an O. sativa or O. glaberrima FatB gene.

43. The method according to claim 39, wherein the biotic stress is selected from the group consisting of rice brown planthopper and rice blast fungus.
